(19) United States
(12) Patent Application Publication
(10) Pub. No.: US 2024/0203290 A1
(43) Pub. Date: Jun. 20, 2024

(54) VITRO MODEL DEVICE FOR CORNEA BARRIER

(71) Applicant: CFD Research Corporation, Huntsville, AL (US)

(72) Inventors: Carrie Lynn German, Huntsville, AL (US); Balabhaskar Prabhakarpandian, Madison, AL (US); Ketan Harendrakumar Bhatt, Huntsville, AL (US)

(21) Appl. No.: 18/541,346

(22) Filed: Dec. 15, 2023

Related U.S. Application Data
(60) Provisional application No. 63/387,566, filed on Dec. 15, 2022.

(51) Int. Cl. G09B 23/30

(52) U.S. Cl. CPC G09B 23/303

(57) ABSTRACT

A microfluidic in vitro cornea device is provided, which can include a tear flow chamber, stromal chamber, endothelial chamber, and aqueous humor chamber. The stromal chamber is adjacent to and porously coupled with the tear flow chamber. The endothelial chamber is adjacent to and porously coupled with the stromal chamber. The aqueous humor chamber adjacent to and porously coupled with the endothelial chamber. A first porous wall is positioned between the tear flow chamber and the stromal chamber. A second porous wall is positioned between the stromal chamber and the endothelial chamber. The third porous wall is positioned between the endothelial chamber and the aqueous humor chamber. The device is configured as a microfluidic in vitro model of a cornea.

### CROSS-REFERENCE TO RELATED APPLICATIONS

This patent application claims priority to U.S. Provisional Application No. 63/387,566 filed Dec. 15, 2022, which provisional is incorporated herein by specific reference in its entirety.

### U.S. GOVERNMENT RIGHTS

This invention was made with government support under 1R43EY034422-01 awarded by the National Institutes of Health. The government has certain rights in the invention.

### BACKGROUND

### Field

This invention relates to the fields of physiology and microfluidics as related to the cornea. More specifically, the present invention pertains to microfluidic systems that mimic the structure, dimensions, fluid flow and physiological function of the corneal barrier as well as complimentary computational models of the microfluidic system and cornea and whole eye.

### Description of Related Art

Over 117 million Americans used eye drops and eye wash in 2020 according to a recent analysis by Statista based on data from the US Census and Simmons National Consumer Survey. It is well known that less than 5% of topically applied medications penetrate the corneal barrier to reach the aqueous humor, yet topical application remains the administration route of choice due to ease and patient compliance. As such, drug development has focused on developing novel carriers and penetration enhancers that promote transport through the cornea and/or improve residence time in the eye.

The cornea functions as a significant barrier to the penetration of foreign substances, such as environmental, industrial, or therapeutic compounds. To cross the corneal barrier, compounds must penetrate 5-7 stratified epithelial layers (e.g., approximately 50 μm thick)¹, a thick stromal layer (e.g., 478-500 μm thick)² and an endothelial layer (e.g., approximately 5 μm thick)³. There are acellular layers on either side of the stroma, which are the Descemet's membrane (e.g., innermost) and Bowman's layer (e.g., outermost). Fluid flows on either side of the cornea: i) as tear film secretion/drainage over the epithelium; and ii) as aqueous humor secretion/drainage over the endothelium. In addition to drainage, effective crossing of the cornea is challenged by the alternating lipophilic, hydrophilic, and lipophilic nature of the sequential corneal layers and the presence of tight junctions in the corneal epithelium. While a compound may easily pass through one corneal layer, it may end up sequestered or rejected from the next layer. Thus, understanding how and to what extent compounds cross the barrier, as well as the effects of penetration enhancing excipients or novel carriers is critical to safely and effectively treating ocular disease and/or trauma as well as vehicle design.

Animal models are commonly used for evaluating corneal penetration⁴. While valuable in vivo data and insights can be obtained via animal models, these tests are expensive and time consuming. Translation of animal model results to humans is often challenging due to species-specific differences in anatomy and physiology. Human corneal explants can help to circumvent issues associated with interspecies differences; however, these explants are typically needed for transplants and are not always readily available for research purposes.

In vitro models incorporating human cells provide an excellent alternative to corneal explants and animal models. Additionally, a wide variety of in silico models have been developed in an effort to predict species-specific and compound-specific pharmacokinetics and corneal barrier penetration. In silico approaches can be used in combination with preclinical models to provide a more powerful tool for prediction of safety, efficacy, bioequivalence, toxicity and more.

In summary, a standardized and validated model or in vitro-in silico framework for evaluating, quantifying, and predicting compound transport through the cornea, distribution in the cornea and pharmacodynamic/toxicodynamic responses does not exist. However, such a standardized and validated model or in vitro-in silico framework is desirable.

### SUMMARY

In some embodiments, a microfluidic in vitro cornea device can include: a tear flow and corneal epithelium chamber (referred to herein as the tear flow chamber); a stromal chamber adjacent to and porously coupled with the tear flow chamber; a corneal endothelial chamber adjacent to and porously coupled with the stromal chamber; and an aqueous humor chamber adjacent to and porously coupled with the corneal endothelial chamber. A first porous wall is positioned between the tear flow chamber and the stromal chamber, a second porous wall is positioned between the stromal chamber and the endothelial chamber, and a third porous wall is positioned between the endothelial chamber and the aqueous humor chamber. The device is configured as a microfluidic in vitro model of a cornea. In some aspects, the tear flow chamber is shaped as an arced microfluidic structure (e.g., chamber, channel, etc.) that is configured to be coupled to a tear flow fluidic network with one or more pumps and optionally one or more media reservoirs. In some aspects, the aqueous humor chamber is shaped as a domed microfluidic structure that is configured to be coupled to an aqueous humor circulation fluidic network with one or more pumps and optionally one or more media reservoirs. In some aspects, the stromal chamber is shaped (e.g., chamber, channel, etc.) as an arced microfluidic structure, which may optionally be configured to be coupled to a stromal fluidic network with one or more pumps and optionally one or more media reservoirs. In some aspects, the endothelial chamber is shaped as an arced microfluidic structure (e.g., chamber, channel, etc.), which may optionally be configured to be coupled to a corneal endothelium fluidic network with one or more pumps and optionally one or more media reservoirs.

In some embodiments, the microfluidic in vitro cornea device includes, in order: the tear flow chamber; the first porous wall; the stromal chamber; the second porous wall; the endothelial chamber; the third porous wall; and the aqueous humor chamber. In some aspects, the cornea device is configured with: the tear film chamber having a width in a range from about 20 microns to about 500 microns; a first porous wall having a width in a range from about 20 microns to about 100 microns; the stromal chamber having a width in a range from about 100 microns to about 1000 microns; a second porous wall having a width in a range from about 20 microns to about 100 microns; the endothelial chamber having a width in a range from about 10 microns to about 200 microns; a third porous wall having a width in a range from about 20 microns to about 1000 microns; the aqueous humor chamber having an apex width in a range from about 100 microns to about 5000 microns; and a height in the tear film chamber, stromal chamber, endothelial chamber and aqueous humor chamber in a range from about 10 microns to about 1000 microns.

In some embodiments, the microfluidic in vitro cornea device includes in order: the tear film chamber having an arc length in a range from about 5 millimeters to about 50 millimeters; a first porous wall having an arc length in a range from 5 about millimeters to about 50 millimeters; the stromal chamber having an arc length in a range from about 5 millimeters to about 50 millimeters; a second porous wall having an arc length in a range from about 5 millimeters to about 50 millimeters; the endothelium chamber having an arc length in a range from about 5 millimeters to about 50 millimeters; a third porous wall having an arc length in a range from about 5 millimeters to about 50 millimeters; and the aqueous humor chamber having a length in a range from about 5 millimeters to about 50 millimeters.

In some embodiments, the widths of the chambers are based on physiological values and the arc lengths determined from the average radius of curvature of the cornea for both healthy and diseased conditions. In some aspects, the chambers can include dimensions that are based on other animals other than humans, such as dogs, cats, pigs, horses, cows, mice, rats, rabbits, or other animals. In some aspects, the chambers can be configured as healthy cornea. In some aspects, the chambers can be configured as diseased cornea. In some aspects, device dimensions or operation thereof can be modified to account for healthy versus diseased cornea (e.g., eye) for any type of animal.

In some embodiments, each porous wall includes a plurality of pore channels that have a width that ranges from about 3 microns to about 8 microns and a height that ranges from about 6 microns to about 10 microns. In some aspects, each pore channel is spaced from about 25 microns to about 75 microns apart from another pore channel.

In some aspects, the aqueous humor chamber includes a plurality of support posts that are configured to provide structural stability under physiological fluidic flow and pressure.

In some embodiments, the microfluidic in vitro cornea device is configured with the following: the tear flow chamber includes corneal epithelial cells; the stromal chamber includes corneal fibroblasts; the endothelial chamber includes corneal endothelial cells; and the aqueous humor chamber includes a fluid that simulates the aqueous humor fluid, which may include endothelial cells or is devoid of cells. In some aspects, the epithelial cells can be primary cells, iPSC-derived cells or a cell line; the fibroblast cells can be primary cells, iPSC-derived cells or a cell line; and the endothelial cells can be primary cells, iPSC-derived cells or a cell line. In some embodiments, the device can include a co-culture or tri-culture of the epithelial cells, fibroblasts, and endothelial cells.

In some embodiments, a microfluidic in vitro cornea system includes: the microfluidic in vitro cornea device of one of the embodiments; and at least one pump configured for moving fluid through the microfluidic in vitro cornea device.

In some embodiments, a microfluidic in vitro cornea system includes: the microfluidic in vitro cornea device of one of the embodiments; at least one camera device configured to be positioned to image at least one of the chambers (e.g. tear flow, stromal, endothelial, or aqueous humor); and a computing system operably coupled with the at least one camera device to receive image data. In some aspects, the computing system is configured to obtain data from the at least one camera device and determine at least one trans-cornea transport property of the microfluidic in vitro cornea device or at least one trans-cornea transport property of a test agent. The trans-cornea transport property can be a measurement of inhibition of transport of a test agent across the corneal barrier and the trans-cornea transport property of the test agent is a measurement of traversal of the test agent across the corneal barrier. In some aspects, the computing system includes one or more computer-readable media storing instructions that when executed cause operations that determine the at least one trans-cornea transport property of the microfluidic in vitro cornea device or the at least one trans-cornea transport property of a test agent.

In some embodiments, a method of studying a cornea includes: providing the microfluidic in vitro cornea device of one of the embodiments having epithelial cells in the tear flow chamber, fibroblasts in the stromal chamber, and endothelial cells in the endothelial chamber; determining a first condition of the microfluidic in vitro cornea device at a first time point; determining a second condition of the in vitro cornea device at a subsequent time point; and determining a change in condition of the in vitro cornea device from the first condition to the second condition.

In some embodiments, a method of studying a cornea can include at least one of: measuring a barrier function property of a corneal barrier, the corneal barrier including epithelial cells in the tear flow chamber, fibroblasts in the stromal chamber, and endothelial cells in the endothelial chamber; imaging the tear flow chamber, stromal chamber, endothelial chamber, and/or aqueous humor chamber through a viewing window of the device; viewing images in real time of the tear flow chamber, stromal chamber, endothelial chamber, and/or aqueous humor chamber through a display screen of a computing system; or measuring transport across the corneal barrier of at least one of nutrients, xenobiotics, small molecules, lipids, liposomes, polymers, particles, toxins, antibodies, others, or combinations thereof. The device can include one or more body members of optically clear material, which can form a viewing window. Thus, the viewing window can be any optically clear portion of the device body.

In some embodiments, a method of studying transport of a test agent across a corneal barrier can include: providing the microfluidic in vitro cornea device of one of the embodiments having epithelial cells in the tear flow chamber, fibroblasts in the stromal chamber, and endothelial cells in the endothelial chamber; providing a test agent as input to any chamber selected from the tear flow chamber or aqueous humor chamber; and monitoring trans-corneal transport of the test agent across the corneal barrier.

In some embodiments, a method of studying transport of the test agents can include at least one of: determining an amount of test agent crossing the corneal barrier and comparing the amount of test agent that crossed the corneal barrier with the administered amount of the test agent introduced into the microfluidic in vitro cornea device; sampling the aqueous humor chamber for the test agent and quantifying the transport of the test agent across the corneal barrier into the aqueous humor chamber; or sampling the tear flow chamber for the test agent and quantifying the transport of the test agent across the corneal barrier into the tear flow chamber.

In some embodiments, a method of studying transport of the test agents can include: evaluating barrier function of the corneal barrier: injecting a plurality of different test agents having a plurality of different sizes into the tear flow chamber; imaging the in vitro cornea device; analyzing images of the in vitro cornea device to identify the plurality of different test agents; and determining a size of test agent or size range of test agent of the plurality of test agents located in the tear flow chamber, stromal chamber, endothelial chamber, and/or aqueous humor chamber.

In some embodiments, a method of studying transport of the test agents can include determining at least one of: a size of test agent or size range of test agents capable of transporting from the tear flow chamber across the corneal barrier into the aqueous humor chamber; a lipophilicity of test agent or lipophilicity range of test agents capable of transporting from the tear flow chamber across the stromal chamber and endothelial chamber into the aqueous humor chamber; or a physiological charge of test agent or charge range capable of transporting from the tear flow chamber across the stromal chamber and endothelial chamber into the aqueous humor chamber.

In some embodiments, a method of studying transport of the test agents can include evaluating permeability of the in vitro cornea device by: injecting one or more test agents into the tear flow chamber; imaging the microfluidic in vitro cornea device; analyzing images of the microfluidic in vitro cornea device to identify locations of the test agent at defined time points, and optionally determine amounts of each test agent in each chamber; and determining a permeability of the in vitro cornea device for the one or more test agents.

In some embodiments, a method of studying transport of the test agents can include evaluating whether the test agent modifies permeability or structural integrity or morphology of the corneal barrier by: determining an initial value of a first property of the corneal barrier; introducing the test agent into the microfluidic in vitro cornea device; determining a subsequent value of the first property of the corneal barrier; and determining a difference between the initial value and the subsequent value of the first property of the corneal barrier, wherein the corneal barrier includes at least the epithelial cells of the tear flow chamber and fibroblasts of the stromal chamber and optionally endothelial cells of the endothelial chamber.

In some embodiments, a method of studying transport of the test agents can include determining a health consequence of the test agent modulating the corneal barrier by correlating the difference between the initial value and the subsequent value and a phenotypic state, which phenotypic state may or may not be a disease state or disorder state.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes a top view of an in vitro cornea device having a microfluidic network that includes the tear film epithelium (TFEp) chamber, stromal chamber, endothelial chamber, and aqueous humor chamber.

FIG. 1B includes a top view of an enlargement of a section of the in vitro cornea device that shows the different features thereof.

FIG. 1C includes a top view of a portion of the in vitro cornea device showing a portion of the stromal chamber, endothelial chamber, and aqueous humor chamber, which are separated by the porous walls.

FIG. 1D includes a side view of a schematic representation of the in vitro cornea device of FIG. 1A.

FIG. 1E includes a schematic representation of an in vitro system having an in vitro cornea device and cameras.

FIG. 2 includes a schematic representation of an exemplary system that includes the in vitro cornea device and fluidic components.

FIG. 3A includes an example of a fabricated in vitro cornea device that is filled with fluorescein sodium salt to demonstrate no leakage.

FIG. 3B includes an example of a fabricated in vitro cornea device where the barrier integrity is tested for size exclusion by static no flow conditions with a mixture of 2 μm and 5 μm particles.

FIG. 3C includes an example of a fabricated in vitro cornea device where the barrier integrity is tested for size exclusion by perfusing the stromal chamber with a mixture of 2 um and 5 μm particles.

FIG. 4A includes an image the in vitro cornea device that shows the cells in different regions separated by the porous walls.

FIG. 4B includes an image of the in vitro cornea device that shows the cells in different regions of the device.

FIG. 5A includes a graph that shows the cell viability data of the three different cell types.

FIG. 5B includes a graph that shows the a permeability index of the three different cell types.

FIG. 5C includes a graph that shows the a permeability index of the three different cell types.

FIG. 5D includes a graph that shows the a permeability index of the three different cell types.

FIG. 6 illustrates an example of a computing system that can be used in the computing systems described herein to perform the computer-implemented methods.

FIG. 7 illustrates a workflow for generating an in silico cornea model using data from the in vitro cornea device.

FIG. 8 illustrates a workflow for providing an in silico cornea model that models the in vitro cornea device.

FIG. 9A includes a flow chart that shows steps for Example 1.

FIG. 9B includes a flow chart that shows steps for Example 2.

FIG. 9C includes a flow chart that shows steps for Example 3.

FIG. 9D includes a flow chart that shows steps for Example 4.

FIG. 10 illustrates an embodiment of a cornea compartmental model.

FIG. 11 illustrates an example of a model of the eye, which can be a 2D PBPK model.

FIG. 12 illustrates a reduced-order quasi 3D model, which can be a PBPK model 550.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology provides an in vitro cornea model in a device configuration for use in obtaining in vitro cornea data that correlates with in vivo data of a real cornea (e.g., real cornea data). The in vitro device allows for obtaining in vitro data that is then modeled with a computing system to create an in silico model for a hybrid in vitro-in silico approach. The in vitro cornea device is configured as a microfluidic device with different chambers that cooperatively and accurately represent the complex physiology of the eye around the corneal barrier. The operational configuration of the device contains: i) a tear film epithelium layer that has epithelial cells, such as corneal epithelial cells, which can simulate a tear film/flow; ii) corneal stroma layer that has stromal cells, such as corneal stromal fibroblasts; iii) an endothelium layer that has endothelial cells, such as corneal endothelial cells; and iv) an aqueous humor layer, which can be configured as a fluid sink. The device geometry and flow rate can be modulated, but are physiologically-based (e.g., chambers are sized to corresponding physical size) to ensure relevance of the in vitro data to in vivo cornea conditions. The corneal barrier in the in vitro cornea device can be evaluated for viability, sustainability, and functionality as compared to corneal explant data from literature or obtained from experiments. That is, the in vitro data from the in vitro cornea device can be correlated with in vivo data from a real cornea, which can be used to create an in silico model of the cornea (e.g., in silico cornea model). The corneal barrier includes at least the epithelial cells of the tear flow chamber and fibroblasts of the stromal chamber, and optionally the endothelial chamber. Also, in vivo data can be used.

The in vitro-in silico corneal barrier hybrid framework comprises: i) a microfluidic in vitro model that recapitulates the corneal environment; ii) a multi-physics-based in silico model of the in vitro cornea device; iii) physiologically-based (PB) high-resolution models of the corneal barrier and/or whole eye; and iv) physiologically-based high-resolution eye models integrated with a physiologically-based whole body (PBWB) model.

The microfluidic in vitro corneal model provides a microfluidic chip comprising a corneal barrier architecture with flow channels that have key physiological dimensions, and a pump system that provides flow rates that yield flow and transport patterns similar to those found in the in vivo cornea environment. A porous architecture separates the channels/chambers, but allows communication via several, repetitive micronized gaps. Barrier integrity can be evaluated by permeability or resistance/impedance. Following exposure/insult, impact to barrier function can be evaluated and compared to normal conditions.

The in silico cornea device model, generated from the in vitro cornea device, comprises a 3D rendering of the microfluidic in vitro device geometry with exact dimensions and a mesh (e.g., structured quadrilateral mesh). Computational domains of the in silico cornea model are specified identical to those described for the in vitro cornea device, such as the tear flow chamber (e.g., epithelium with tear film), stromal chamber, endothelial chamber, and aqueous humor chamber. Fluid flow and species-transport are characterized by the convection-diffusion equation and solved simultaneously using a multi-physics solver. Active transport is included using sink/source terms in the respective compartments.

The PBWB models are in silico models comprised of multiple compartments representing the organs/organ systems in the body of a human or other animal. Model parameters, including organ volumes, blood flow rates, metabolic parameters and others are species-dependent. Species transport and elimination are modeled using a system of ordinary differential equations, which are simultaneously solved using a multi-physics model solver. In some aspects, the in silico cornea model can be configured as an integrated corneal-PBWB model.

In some embodiments, the in vitro corneal model can be correlated to a corresponding in vivo cornea model. This allows the in vitro cornea data to be used to develop corneal physiologically-based (PB) pharmacokinetic (PK) models, which can be connected through a high-resolution in silico cornea model. The corneal PBPK model can be adapted using software, such as CoBi tools. The PBPK in silico cornea model combined with the in vitro cornea device enables the prediction of corneal PK values for a range of test agents, such as those that are pharmaceutically relevant. For example, data obtained from in vitro experiments using the in vitro cornea device described herein can be used to characterize drug transport at the level of the whole cornea. Such an in vivo and in silico hybrid cornea model can be obtained by developing a first-principles based model of the in vitro corneal barrier based on the in vitro cornea device to evaluate the predictive capability for use as a cornea transport model. The in silico cornea model can then be scaled up to the level of the whole cornea. As such, a scaling factor can be determined based on the data of the in vitro cornea device and real cornea data, in order to correlate the in vitro cornea data with real cornea data. The in silico cornea model can account for diffusive and active transport of a range of test agents. The development of this hybrid cornea model as a diagnostic and monitoring platform can aide in the prediction of impact of a chemical passing through the cornea and the downstream health effects in the eye, and whole body of humans and animals.

The invention addresses the need for a standardized platform that improves understanding of the corneal barrier and enables prediction of corneal drug transport in vivo without actually using a real cornea. This study combines modeling approaches to create a full understanding of the eye or corneal exposure to drugs taken by application onto the eye or accidental exposure to other compounds. These studies can aide in the safe and effective treatment of the medical needs of the eye, enabling the provision of guidelines for prescription of medications, and open up the possibility of therapeutically treating eyes or whole body by ocular administration.

The combination of the in vitro cornea device and the computer-generated in silico cornea model provides the in silico-in vitro hybrid cornea system that builds upon validated in vitro models of the cornea for predicting exposure levels for different routes of administration and doses of one or more test agents. These computer simulated predictions are valuable because corresponding studies cannot be performed clinically, and in vitro cornea data for many agents can be lacking. The combination of the in silico-in vitro hybrid cornea system allows for predicting the pharmacokinetic profiles of a test agent (e.g., drug) in the aqueous humor, eye tissues, or at the corneal barrier. This hybrid cornea system allows for the in silico cornea model to generate synthetic in silico cornea data that can be used in determining appropriate doses for the safe treatment of an eye or whole body via ocular administration.

The synergistic in vitro-in silico hybrid cornea model can be used for: i) evaluating compound transport of a test agent across the corneal barrier and ii) predicting eye and whole body compound exposure and toxicity of the test agent. The microfluidics-based corneal barrier platform provided by the in vitro cornea device provides physiologically relevant data while enabling real-time morphological, pharmacokinetic and toxicological evaluations. For example, an imaging system, or any other assay system can be operably coupled with the in vitro cornea device so as to be able to obtain real time data thereof. The in silico model of the cornea can be generated based on the microfluidic in vitro cornea device platform. The in vitro and in silico hybrid cornea model can be used for predicting drug and nutrient transport based on compound physicochemical properties and tissue properties in combination with physics-based transport laws. This work builds upon previously validated and commercially available microfluidic 3D tissue platforms (e.g., Syn Vivo) and Computational Biology (CoBi) tools, which can be used to model physiological interactions.

Both the in vitro cornea device and in silico PBPK cornea model are based on ocular anatomy and physiology collected from literature. All in vitro model dimensions are based on in vivo measurements. Physiological relevance is established by the presence of organ specific cell cultures, which can be generated by the cells in the in vitro model. For in silico PBPK and in vitro hybrid cornea model integration, a schematic that demonstrates the validated approach for the connection between in silico (e.g., computer compartmental model of cornea), in vitro cornea device, and in vivo cornea data is provided. The modeling of the in vitro cornea device itself is performed and compared to in vivo cornea data. The system allows for obtaining passive and active transport parameters from successive experiments, which can be scaled based on surface area or transporter expression to full-size cornea and act as input to the in silico cornea model. The PBPK in silico cornea model results can be compared with PK profiles from explants, in vivo studies and in vitro cell culture experiments to demonstrate validity. The scaling can take the size and data of the in vitro cornea device and map it to an in vivo cornea model, and a scaling factor or scaling function can be determined which can then be used to generate the synthetic in silico cornea data.

Cell morphology, proliferation and tight junction functionality are all endpoints that can be studied to determine whether or not they are dependent on a suitable ECM for the in vitro cornea device. Accordingly, the cells and ECM in each compartment of the in vitro cornea device can be configured to mimic the corresponding in vivo structure and condition of health or disease. The in vitro data is improved by using relevant physiological geometry and dimensions derived from in vivo architecture. In addition, physiological fluid flow values are used to drive controlled perfusion in the in vitro cornea device. Also, quantitative values for biomarkers can be used to validate the in vitro model relevance by comparison of those same biomarkers.

The cornea barrier function can be evaluated in a number of ways. The in vitro cornea device can be used to evaluate real cornea barrier function by validating cornea barrier function of the in vitro device by exclusion of a control substance (e.g., trypan blue) from the aqueous humor chamber, which can be evaluated by mass spectrometry. Also, the extent to which other control substances (e.g., 4 kD FITC-dextran, fluorescein) permeate the corneal barrier can be compared to data collected from corneal explants and the in vitro cornea device's cell culture for the same substances. Transport parameters can be normalized by surface area for the in vitro model, cell culture (well plate) model, explant models, and in vivo data. Also, the system can evaluate glucose transport, which is facilitated by diffusion via hexose transporters (e.g., GLUT3 and GLUT1).

Accordingly, the in vitro cornea device can be validated using a known test agent for comparison with data for that known test agent in a real cornea. The correlation between in vitro cornea data and real cornea data provides a basis for studying various test agents with the in vitro cornea device described herein, so that the in silico cornea model can be used for these various test agents to generate relevant cornea data without using a real cornea.

### In Vitro Cornea Device

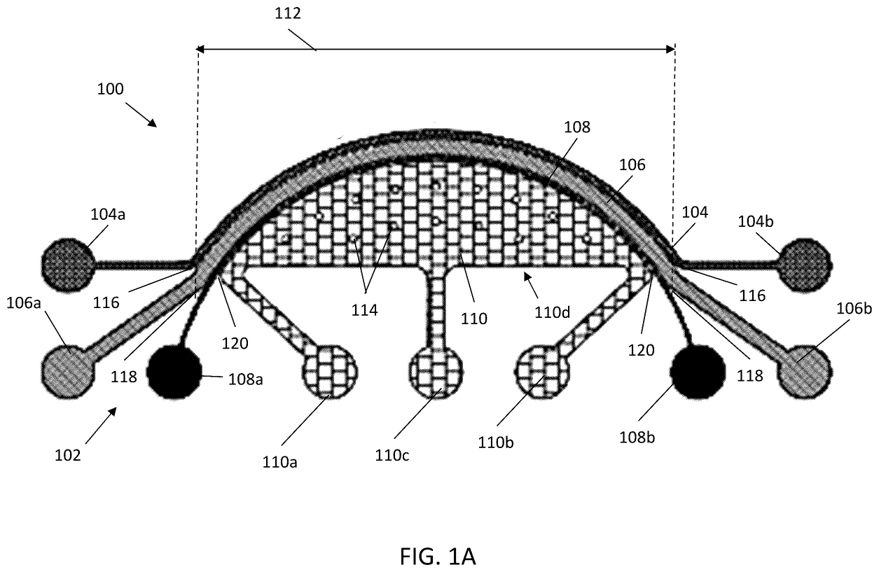

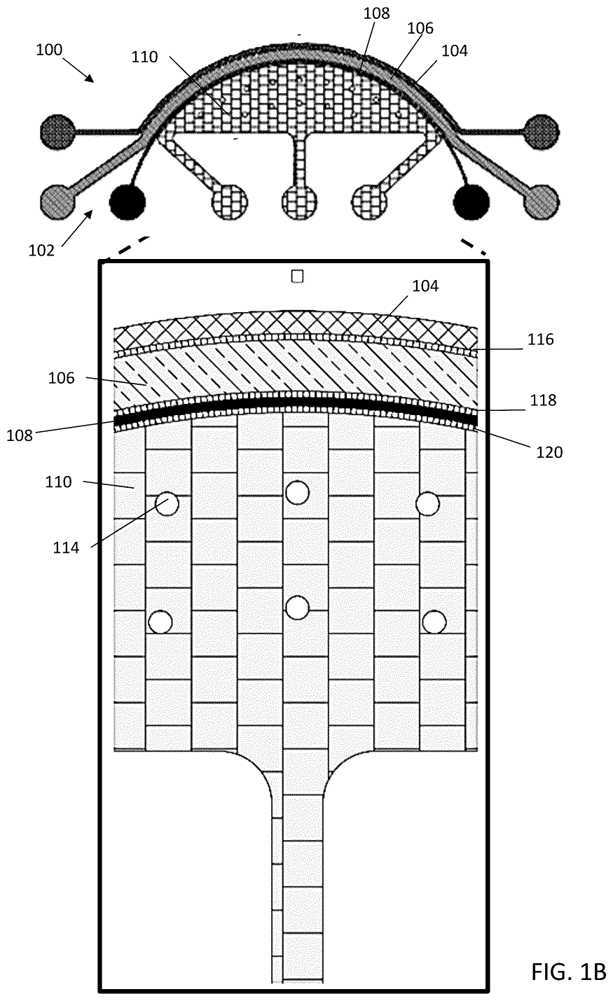

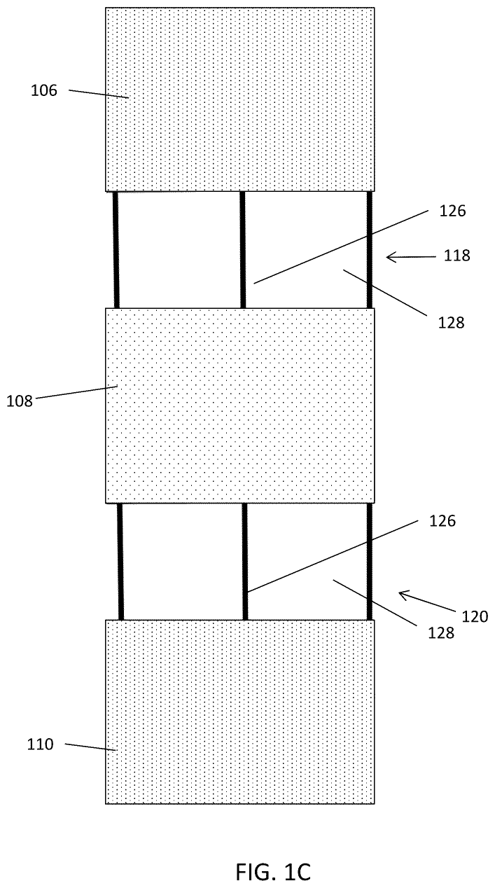

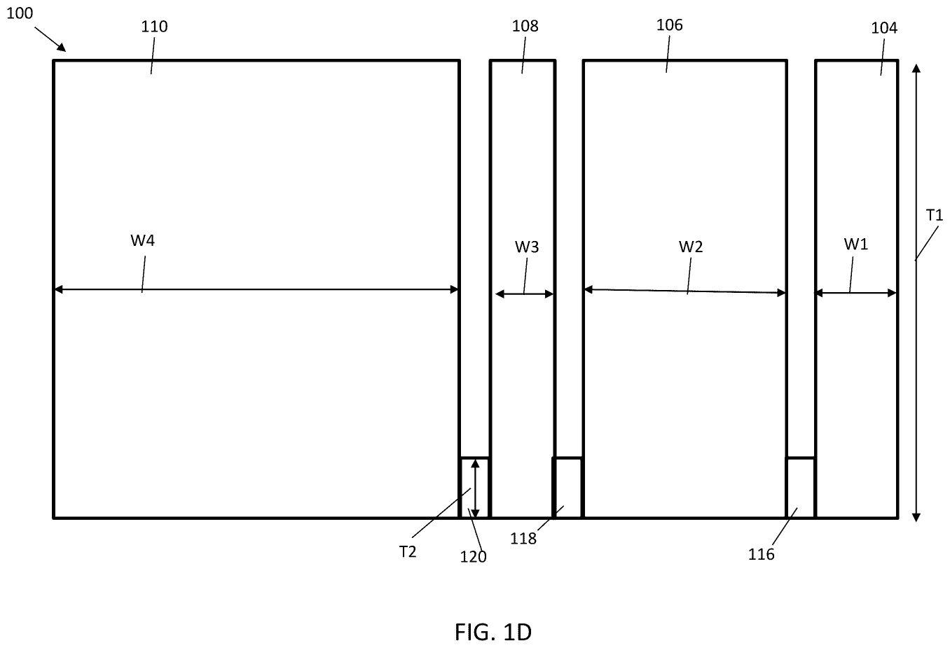

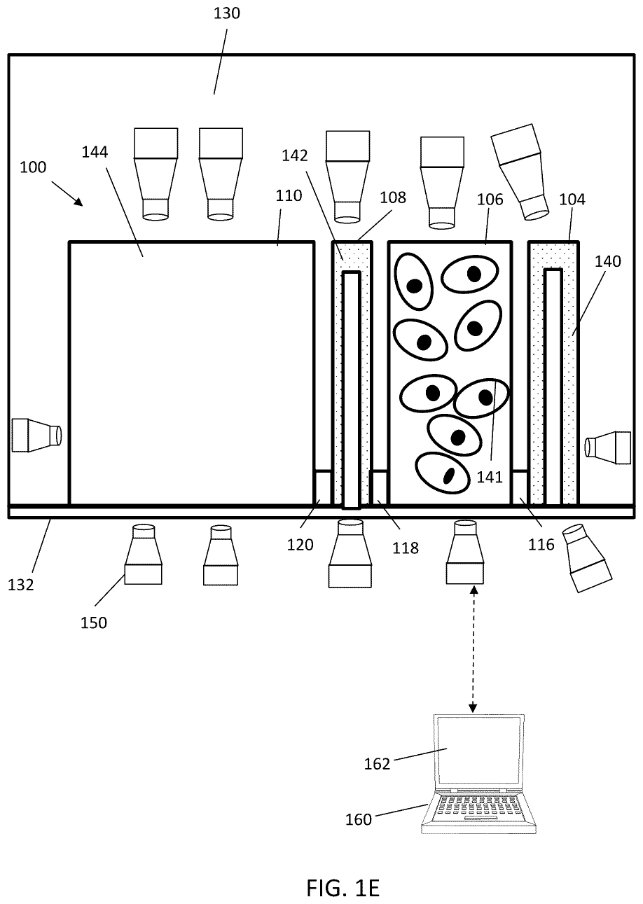

An embodiment of the in vitro cornea device 100 is shown in FIGS. 1A-1E. FIG. 1A includes a top view of the in vitro cornea device 100 having the microfluidic network 102 that includes the tear film epithelium (TFEp) chamber 104, stromal chamber 106, endothelial chamber 108, and aqueous humor chamber 110. The TFEp chamber 104 is shown to include an inlet 104a and an outlet 104b, with the cell culture region therebetween, which is the region between the marker lines for the assay region 112. As shown, the assay region 112 can be about 10 mm to 20 mm across with the arc, but can vary as described herein. The stromal chamber 106 is shown to include an inlet 106a and an outlet 106b, with the cell culture region therebetween, which is the region that is arced for the assay region 112. Accordingly, the TFEp chamber 104, stromal chamber 106, and endothelial chamber 108 may include a cell culture of stroma fibroblast cells with or without extracellular matrix material (e.g., natural or synthetic), or other biological molecules or hydrogels. The endothelial chamber 108 is shown to include an inlet 108a and an outlet 108b, with the cell culture region therebetween, which is the region between the marker lines for the assay region 112. The aqueous humor chamber 110 is shown to include an inlet 110a and an outlet 110b as well as a central port 110c, with a sink region 110d (e.g., with or without cells) therebetween, which is the dome region between the marker lines for the assay region 112. The aqueous humor chamber 110 is shaped as a partial circle (e.g., dome), where the TFEp chamber 104, stromal chamber 106, and endothelial chamber 108 are arced to follow the curvature of the curved side of the aqueous humor chamber 110, which mimics the shape of the cornea and associated regions of the eye. The size and shape may simulate an eye curvature. Additionally, structural posts 114 are shown in the aqueous humor chamber to provide structural support to the open area.

The chambers are separated by porous walls that form an interface between adjacent chambers. A first porous wall 116 separates the TFEp chamber 104 from the stromal chamber 106, a second porous wall 118 separates the stromal chamber 106 and the endothelial chamber 108, and a third porous wall 120 separates the endothelial chamber 108 and the aqueous humor chamber 110. Notably, a plurality of the in vitro cornea devices 100 can be used together in a system, such as in an any series or parallel, or combination thereof.

FIG. 1B includes a top view of an enlargement of a section of the in vitro cornea device 100 that shows the different features thereof. As shown, the arrangement includes in order: the outer TFEp chamber 104; the first porous wall 116; the stromal chamber 106; the second porous wall 118; the endothelial chamber 108; the third porous wall 120; and the aqueous humor chamber 110. The porous walls, 116, 118, 120 are shown to have pore channels 126 separated by wall sections 128, which are further illustrated and labeled in FIG. 1C.

FIG. 1C shows a top view of a portion of the in vitro device 100 showing a portion of the stromal chamber 106, endothelial chamber 108, and aqueous humor chamber 110, which are separated by the porous walls (e.g., 118, 120) that each have the individual pore channels 126 in the porous walls. The pore channels 126 can have a width of 5 microns, but can vary as described herein. The pore channels 126 can be separated from each other by a wall sections 128, which can have a width of 50 microns, but can vary as described herein.

FIG. 1D shows a side view of a schematic representation of the in vitro cornea device 100 of FIG. 1A. As shown, the TFEp chamber 104 has a width W1 across of 20 microns to 500 microns, the stromal chamber 106 has a width W2 of 100 microns to 1000 microns, the endothelial chamber 108 has a width W3 of 10 microns to 200 microns, and the aqueous humor chamber has a width W4 at the apex of the dome of 100 microns to 2000 microns. The TFEp chamber 104, stromal chamber 106, endothelial chamber 108, and aqueous humor chamber 110 can all have a thickness T1 (e.g., height) of 100 microns. FIG. 1D also shows that the porous walls 116, 118, 120, all have a width of 50 microns and a thickness T2 of 8 microns. However, these dimensions can be varied as described herein.

FIG. 1E shows a substrate 130 having the in vitro cornea device 100 formed therein, and a glass slide 132 (or other lid) on a side. The TFEp chamber 104 is shown to have a corneal epithelial cell culture 140. The stromal chamber 106 is shown to include the cell culture to simulate the stromal layer of the cornea barrier that includes fibroblasts 141. The endothelial chamber 108 is shown to have an endothelium cell culture 142. The TFEp chamber 104, stromal chamber 106, and/or endothelial chamber 108 can include a extracellular matrix, and other components (e.g., proteins) to mimic physiological conditions. The aqueous humor chamber 110 is shown to include an aqueous fluid 144. The substrate 130 can be PDMS or other biocompatible structural material. The glass slide 132 can be glass or plastic, such as PDMS. The stomal chamber 106 can have an extracellular matrix secreted by the cells therein.

The microfluidic in vitro corneal barrier model is shown to include four fluidic channels separated by three porous barriers in a side-by-side architecture. The porous barrier between the channels has 5 μm slit pores spaced 50 μm apart from each other, which permits cell-cell communication and diffusion of signaling molecules and various chemical compounds. The aqueous humor chamber is reinforced by 200 μm diameter support posts 114 to avoid collapsing under fluidic flow and pressure. Unlike traditional microfluidic devices, channel thicknesses can be selected based on dimensions of the human corneal environment of an adult or child., as well as an animal of any age.

The in vitro cornea device can include a disposable and optically clear microfluidic chip containing 3D cultured stromal cells (e.g., fibroblasts, keratocytes), epithelial cells, and endothelial cells, in physiological dimensions that accurately capture in vivo architecture. The in vitro cornea device more accurately reproduces the in vivo microenvironment, which provides a physiologically relevant testing system for test agent exposure and toxicity analysis. The configuration of the device and use of PDMS (polydimethylsiloxane) that is optically clear provides the ability for real-time, high content, quantitative imaging of cell and test agent interactions, as well as the responses of cultures to drug/toxin exposure and biological secretions. In some aspects, the device employs a native cornea-derived extracellular matrix (ECM) for optimal and realistic culture conditions enabling physiologically relevant behavior.

FIG. 1E also shows cameras 150 that can be placed outside of the glass slide 132, outside the substrate 130, or embedded in the substrate 130 (e.g., in etched regions). The cameras 150 can be communicatively coupled with a computing system 160 configured as a controller and to receive optical data from the cameras 150. The cameras 150 can take still images or videos of the different chambers of the in vitro cornea device 100. While a number of cameras 150 are shown in an arrangement, the placement and number of cameras can be modified in order to obtain the desire data, where more or fewer cameras can be used. As such, the entire device or select regions of interest can be imaged with the imaging system. The computing system 160 can be communicatively coupled with the cameras 150 by wire, optics, or wireless communication networks, represented as the dashed lines. The computing system 160 can include a display 162 for visually showing the images obtained from the cameras 150.

The invention comprises a device, which can be referred to as an in vitro cornea device, lab-on-a-chip cornea device, or cornea-on-a-chip device, designed for the purpose of analyzing a biological structure of a cornea. This device is composed of several interconnected systems and components.

The first main component is the TFEp system, configured as a microvascular network or microchannel network. This system is designed to mimic the ocular tear flow for a real cornea. It is connected to a microfluidic network with one or more pumps (e.g., microfluidic pumps or micropumps), and optionally one or more reservoirs, such as a cell culture media reservoir, test agent reservoir, positive control reservoir, or negative control reservoir. The output from this system is fluid that is directed towards the next component, the stroma. The pumps can include peristaltic pumps, syringe pumps, or gravitational flow driven mechanisms that cause fluid flow.

The stromal chamber, also referred to as the corneal stroma, is designed to mimic the space in a real cornea where the stroma cells reside. This space is also connected to a network and devices, such as the TFEp system. The output from this space is fluid that is directed towards the next component, the endothelium.

The endothelial chamber is designed to mimic the endothelial layer in a real cornea that separates the stroma cells from the aqueous humor fluid (e.g., 144) while allowing the exchange of nutrients and waste. The endothelial chamber is also connected to the same fluidic network and devices as the previous components. The output from the endothelium is fluid that is directed towards the final main component, the aqueous humor circulation system. For example, the aqueous humor fluid can be saline, cell culture media, or artificial aqueous humor fluid.

The aqueous humor chamber and system is designed to mimic the fluid in the aqueous humor, from flow to volumetric sink, such as is present in a real eye. It is connected to the same fluidic network and devices as the previous components. The output from this system is fluid that is directed towards collection devices in the fluidic network, such as waste collection devices.

Each of these main components and their sub-components work together to enable the device to accurately mimic the structure and function of a real cornea, allowing for detailed analysis and study. The device also includes a viewing window, or observation window, which provides visibility into the systems and spaces. The viewing window can, facilitate the monitoring and examination of the processes occurring within the device. The windows can be used with cameras to record the processes occurring within the device, such as transport of test agents, or changes in cell morphology. For example, a clear device body can be used as the viewing window.

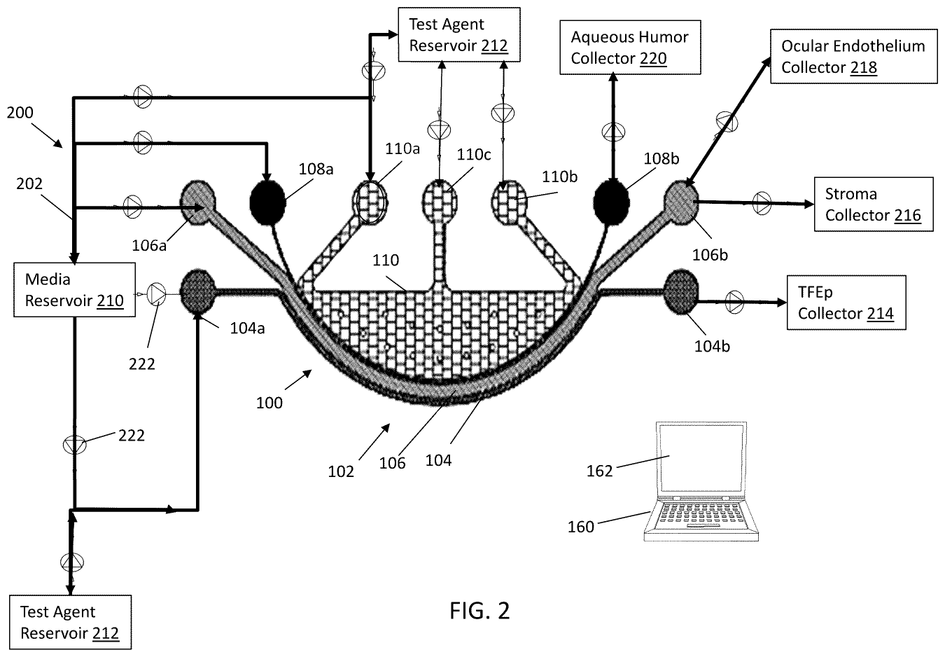

FIG. 2 shows an exemplary system 200 that includes the in vitro cornea device 100. As shown, a fluidic network 202 including a media reservoir 210 feeding the TFEp chamber 104, stromal chamber 106, endothelial chamber 108, and/or the aqueous humor chamber 110, which can include one or more pumps 222 to facilitate fluid flow. However, each chamber may have its own media reservoir, which can include a specialized media configured for the cell culture therein, such as growth factor, or other biomolecules. Also, a test agent reservoir 212 is shown to feed into the TFEp chamber 104 and/or the aqueous humor chamber 110. The test agent reservoir 212 can include test agents to be used in the methods described herein on monitoring the endothelium and transport properties thereof. Any number of test agent reservoirs 212 can be used for any number of test agents. The test agents can be any type of test agent, such as those described herein.

FIG. 2 also shows the outlet of each chamber coupled to a particular collector, which can include one or more pumps 222 to move the fluid. The pumps 222 can be used to precisely control the fluid flow through the channels and chambers. The TFEp chamber 104 is connected to a TFEp collector 214 adapted to collect the fluid from the outlet 104b. The stromal chamber 106 is connected to a stroma collector 216 adapted to collect the fluid from the outlet 106b. The endothelial chamber 108 is connected to an endothelium collector 218 adapted to collect the fluid from the outlet 108b. The aqueous humor chamber 110 is connected to an aqueous humor collector 220 adapted to collect the fluid from the outlet 110b. Various valves or other fluidic network components can be included, such as heaters, coolers, or the like. The components of the system 200 can be controlled by the computing system 160.

The microfluidic in vitro cornea devices can be fabricated using standard PDMS soft-lithography techniques as known. CAD drawings of the microfluidic corneal barrier device geometry can be generated to create SU-8 silicon molds, and device architecture is realized by casting with PDMS. Inlet and outlet ports are punched into the PDMS mold and then the structure is bonded to clean glass slides to form the final microfluidic chip prototypes. Alternatively, the glass slides can be configured as lids or covers for the microfluidic network.

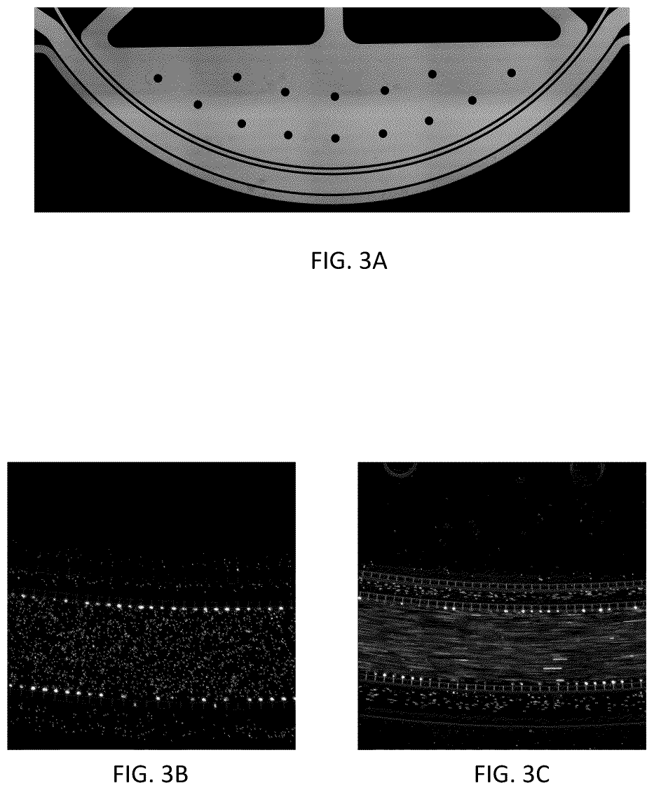

The integrity of the device and porous barriers can be evaluated by perfusion with fluorescent dye or particles. FIG. 3A shows an example where a fabricated device is filled with fluorescein sodium salt to demonstrate no leakage. FIG. 3B shows another example, where the barrier integrity is tested for size exclusion by static no flow conditions with a mixture of 2 μm and 5 μm particles. FIG. 3C shows another example, where the barrier integrity is tested for size exclusion by perfusing the with a mixture of 2 μm and 5 μm particles. The smaller particles freely diffuse across the slit barrier, while the larger particles are retained, indicating a fully functional 5 μm porous architecture.

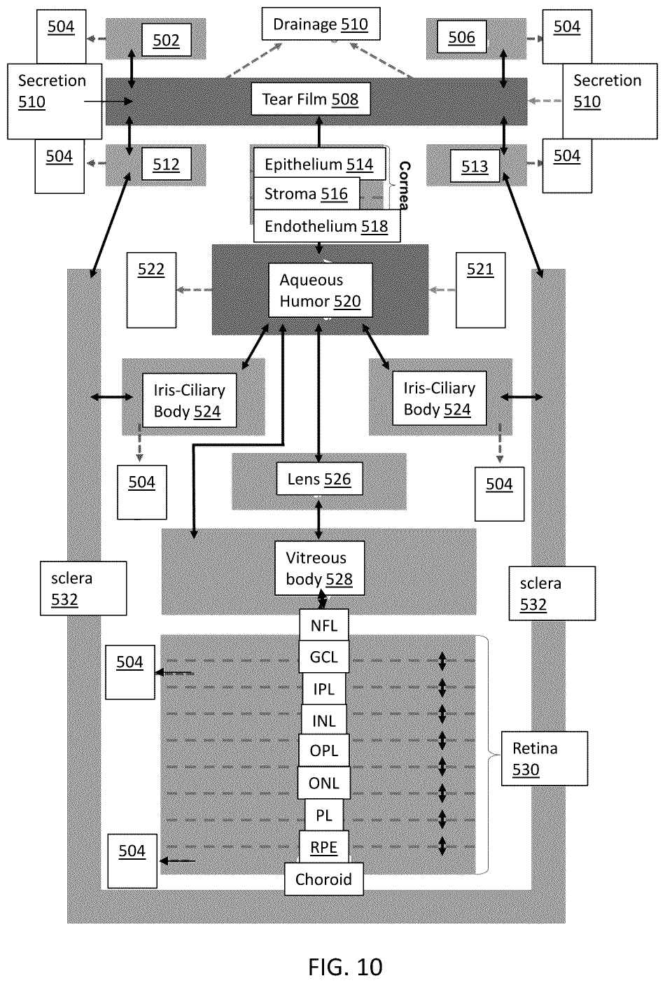

FIG. 10 illustrates an embodiment of a whole eye compartmental model 500, which is a PBPK model that includes the cornea. The whole eye compartmental model 500 includes: PC_{L} (Lower palpebral conjunctiva) 502 to plasma 504; PC_{U} (Upper palpebral conjunctiva) 506 to plasma 504; tear film 508 receiving secretion 510 and providing drainage 512; BC_{L} (Lower bulbar conjunctiva) 512 to plasma 504; BC_{U} (Upper bulbar conjunctiva) 513 to plasma 504; epithelium 514; stroma 516; endothelium 518; aqueous humor 520 that receives aqueous humor secretion 521 and provides aqueous humor drainage 522; iris-ciliary body 524 to plasma 504; lens 526; vitreous body 528; retina 530 to plasma 504; and sclera 532. Species transport is shown by the arrows, where dashed arrows represent fluid flow and solid arrows represent diffusive transport. Similar to the PBWB model, each tissue of the eye is represented as an individual compartment with its own properties (e.g. viscosity, density, metabolic constants, etc). Species transport is characterized by a system of ordinary differential equations describing both fluid flow- and diffusion-based contributions.

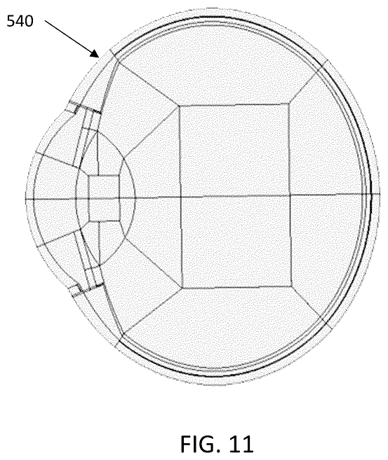

FIG. 11 illustrates an example of a high-resolution model of the eye, which can be a 2D PBPK model. The model is a representative slice taken through the middle of the eye. In this embodiment, the cornea is represented as single compartment rather than multiple layers. The high-resolution model includes spatial resolution that can consider local concentration gradients that drive and effect species transport. Fluid flow and species transport are coupled and characterized by a system of partial differential equations. The highly resolved nature of the model makes it computationally expensive.

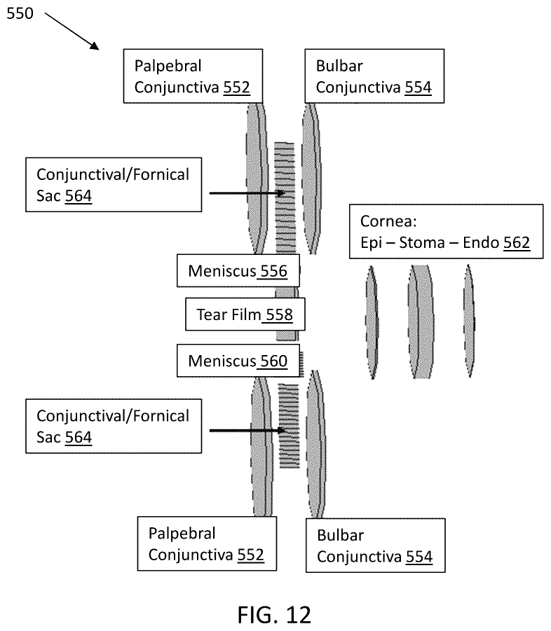

FIG. 12 illustrates a reduced-order quasi 3D model 550. The reduced-order quasi 3D model 550 is shown to include the palpebral conjunctiva 552 and bulbar conjunctiva 554, and with the conjunctival/fornical sac 564, along with the meniscus 556, tear film 558, and meniscus 560, as well as the cornea 562 with the epi (epithelium), stroma, and endo (endothelium). A compromise between the simplicity of the compartmental model and the complexity of the high-resolution model, the reduced order model is spatially resolved in the direction of fluid flow. Partial differential equations are used to characterize fluid flow and species transport. The reduction in dimensionality makes this approach less computationally expensive than traditional 2D and 3D models.

Pharmacokinetic/toxicokinetic and pharmacodynamic/toxicodynamic parameters (e.g. permeability rate, active transport rates, binding kinetics, and others) determined from experiments using the microfluidic in vitro cornea device and in silico model of the microfluidic in vitro cornea device can be scaled and used as inputs for the cornea PBPK model examples shown in FIGS. 10-12. These whole eye PBPK models can then be used to generate synthetic PK profiles for prediction of whole eye PK for comparison with in vivo data. Just as the in vitro cornea device can be modified to represent different states of health and diseases as well as different species, so can the whole eye PBPK models.

### In Vitro Cornea Device Coatings and Cultures

The in vitro corneal model devices are primed with sterile phosphate buffer solution (PBS) by injection into each of the channels. All chambers of the microfluidic corneal device may be coated with various proteins or substrates, creating an extracellular matrix to support the attachment and growth of cells on inner surfaces of the chambers. Example substrates include, but are not limited to, fibronectin, collagen and lyophilized corneal extracellular matrix (ECM). This could also include any combination of natural and/or synthetic matrices to represent healthy or diseased ECM. The methods for coating various surfaces (e.g., glass, plastic) with proteins and other substrates are well known in the field. Cells, such as corneal epithelial cells, stromal cells (e.g., stomal fibroblasts), and endothelial cell, may be cultured on the coated inner surfaces of the relevant chambers to study species transport from TFEp circulation into the cornea. Virtually any type of epithelial (e.g., corneal epithelial cell) or endothelial cell (e.g., corneal endothelial cells (CEnC), corneal endothelial cells, etc.) may be cultured in the microfluidic corneal devices. These cells may also be cocultured or tricultured.

In some embodiments, all channels of the device can be coated with human fibronectin to facilitate cellular attachment. For example, primary human corneal fibroblasts can be seeded on Day 0. Fibroblasts can be harvested, concentrated to 1×10⁷ cells/mL and injected into the stromal chamber via a remote head syringe pump at 5 μL/min until cells are homogenously distributed. Devices can then be incubated at 37° C., 5% CO₂ for 4 hours to allow for attachment. Media can be subsequently refreshed every 24 hours. Primary human corneal epithelial cells can be seeded into the TFEp circulation channel on Day 3. Epithelial cells can be harvested, concentrated to 2×10⁷ cells/mL and injected into the TFEp circulation channel via a remote head syringe pump at 5 μL/min until cells are homogenously distributed. Devices can then be incubated at 37° C., 5% CO₂ for 4 hours to allow for attachment. The representative tear flow protocol is subsequently initiated in the TFEp circulation channel. Immortalized human corneal endothelial cells can be seeded into the endothelial chamber on Day 5 at 6×10⁷ cells/mL using a flow rate of 3 μL/min until cells are homogenously distributed. Devices can again be placed in the incubator for 4 hours to allow for attachment. The aqueous humor (AH) channel can be connected to flow 4 hours later. Thus, the devices can be configured with cells and media for use in the cornea assays, such as studying corneal transport with test agents introduced into the TFEp and/or aqueous humor circulation systems for testing corneal penetration.

In some embodiments, an assay can be performed with the in vitro cornea device for characterization of cell cultures in the chambers of the microfluidic network. Cell viability, morphology and cell-cell interactions can be observed microscopically in real-time throughout all experiments. The imaging system can be used to monitor the device so that the cultures can be visualized as shown.

Additionally, end point assays, such as those for viability, phenotypic protein expression, metabolic activity, gene expression, and the like can be assessed on- or off-chip. For example, fluidic samples can be obtained at the outlet of each chamber throughout an assay and/or cell samples can be obtained after an assay for biomarker profile analysis. Along with visualization, a sample analysis assay can be used with samples obtained from the different chambers. The biochemical analysis along with the visual analysis can be useful in modeling the in vitro system.

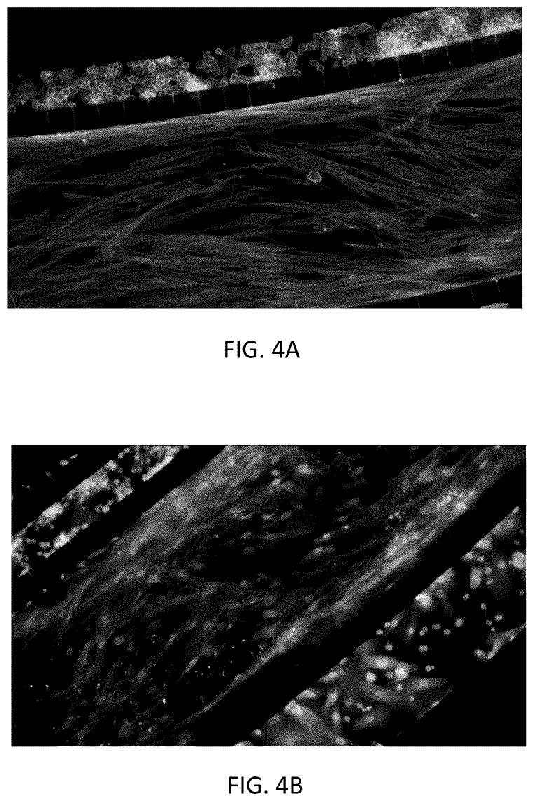

FIGS. 4A and 4B show a phenotypic expression of the different cell cultures in the different chambers of the in vitro cornea device. As shown, FIG. 4A shows the cells in different regions separated by the porous walls. Human corneal endothelial cells are shown in the endothelial chamber (upper left), with human corneal fibroblasts in the stromal chamber (middle) and human corneal fibroblasts in the TFEp chamber (bottom right). Cells are stained for collagen type 1, collagen type 3, and mucin type 1, phenotypic markers for stromal ECM, fibrotic stromal ECM and epithelial cells, respectively. FIG. 4B shows the cells in different regions of the device, focusing on human corneal endothelial cells in the thinner TFEp chamber and human corneal fibroblasts in the wider chamber with cells showing phalloidin staining. Phenotypic expression of each cell type of one iteration of the device and one iteration of the cellular combinations can be confirmed by immunocytochemistry staining. The stains show Col, Col3, and MUC1, and phalloidin for staining actin filaments.

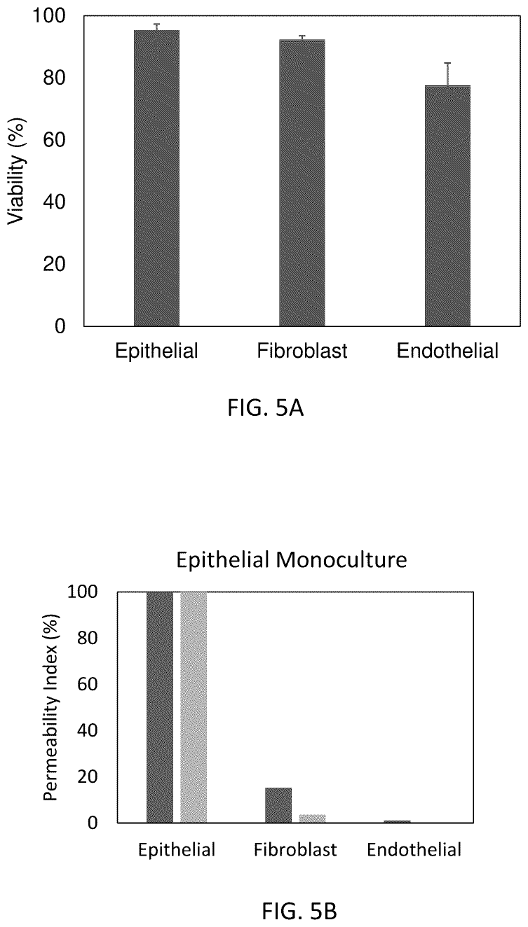

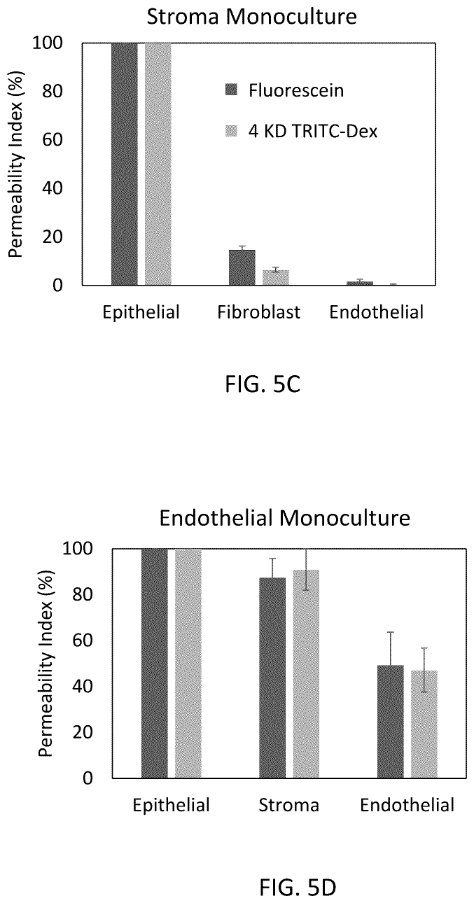

Viability can be assessed on-chip as an endpoint for epithelial, fibroblast, and endothelial cells. Devices are prepared for viability quantitation by incubation with calcein AM and ethidium homodimer in PBS. After rinsing the cells with PBS, fluorescent images are taken via microscope, and the data was analyzed to determine cornea triculture viability (sec, FIG. 5A). Live and dead cells (e.g., epithelial, fibroblast, endothelial cells) are visualized using FITC and TRITC filters, respectively. The graph in FIG. 5A shows the cell viability data of the three different cell types. Viability assessment via a live/dead fluorescent staining kit demonstrates the triculture is highly viable. For example, an in vitro cornea device can be exposed to trypan blue by injection into the TFEp channel at 3 μL/min for 4 min and then incubated at 37° C. for 15 min. The morphological effects of trypan blue exposure can be readily observed at any chamber with evidence of barrier degradation (e.g., cell death and detachment) and apoptosis.

Following exposure, FSS can be injected into the TFEp channel and permeability assayed. Permeability in the devices can be 10-fold higher in trypan blue treated devices compared to controls. An additional assay can be performed to determine cytotoxicity as it relates to the release of adenylate kinase. A positive control (100% death by lysis buffer) and a negative control (media only) can be included for comparison. Samples can be taken from cell-laden channels (individually or combined) and placed in a low volume, white walled 385-well plate for luminescence analysis using a plate reader. Trypan blue exposures are expected to result in a 1.5-fold increase in adenylate kinase release. The increase in AK release in arbitrary luminescence units compares the toxic exposure of trypan blue to positive and negative controls.

FIGS. 5B, 5C, and 5D show the data for assessing the barrier permeability for various culture conditions and demonstrate differences in barrier function by corneal layer using fluorescein and 4 kD TRITC-dextran. Size dependence is also shown. FIG. 5B shows an epithelial monoculture showing the permeability index with regard to epithelial, fibroblast, and endothelial chambers. FIG. 5C shows a stroma monoculture showing the permeability index with regard to epithelial, fibroblast, and endothelial chambers. FIG. 5D shows an endothelial monoculture showing the permeability index with regard to epithelial, fibroblast, and endothelial chambers. In FIGS. 5B-5D, the light bar is for 4 kD TRITC-Dex and the dark bar is for fluorescein. For example, one embodiment of the present invention can be tested for differences in permeability, where a 4 kD TRITC-Dextran solution can be injected into the TFEp channel at 0.1 μL/min for 1 hour. Endpoint images can be taken and the permeability index (PI) can be determined as the ratio of the sum ROIs in the channel of interest compared to the TFEp channel. In another example, one embodiment of the present invention can be used to evaluate corneal size exclusion, where a fluorescein sodium salt (FSS) solution can be injected into the TFEp channel at 0.1 μL/min for 1 hour. Endpoint images can be taken and the PI determined. PI can be expected to decrease as a function of compound molecular weight and corneal layer representation. For example, the corneal epithelium is the greatest barrier to transport and is expected to demonstrate a lower PI in the subsequent channels compared to the corneal endothelium. This study can show size-dependent as well as barrier layer-dependent permeability.

In some embodiments, the in vitro cornea device can be used to model corneal barrier function. The corneal barrier (e.g., including the TFEp and aqueous humor vasculature) functions by preventing specific chemicals from crossing the barrier. The present in vitro cornea device can be used to study mechanisms of exclusion including size, net charge, lipophilicity, active transport, and the like. Differences in aqueous humor-corneal and TFEp-corneal transport have also been described in the literature, which can be compared to the in vitro cornea data. Permeability assays using chemicals with these various physicochemical properties can be conducted to evaluate barrier function of the in vitro cornea device.

In some embodiments, the in vitro corneal device can be used for studying toxicity of test agents to the cornea and to the eye in general once entering the aqueous humor. Certain compounds not only induce corneal or eye toxicity upon crossing the corneal barrier, but also disrupt the barrier itself. The microfluidic corneal model can be used to study response to toxic exposure of test agents. The impact to barrier function can be assessed through permeability comparisons and toxicity (e.g., cytotoxicity, etc.) can be assessed off chip by various methods (e.g., cell lysate luminescence assay, genomic analysis, etc.).

### Modeling and Simulation of Trans-corneal Transport

In some embodiments, the data that is obtained from the in vitro cornea device can be used for modeling and simulating an in vivo cornea in an in silico framework. As such, the data from the in vitro cornea device can be correlated with known in vivo cornea data for different substances and conditions. As such, the in vitro cornea device data can be used to model the in vivo data of a real cornea. The in vitro cornea device can then be used to simulate trans-corneal transport for various types of substances. The model can be tailored for the type of substances that are going to be used for test agents, such as by type, size, charge, and the like.

Some embodiments of the present invention include a computational model for analysis of fluid flow and species transport of test agents in the microfluidic in vitro cornea device. Computational model construction can begin with the generation of a 3D geometry of the microfluidic chambers and porous architecture of the in vitro cornea device followed by the generation of a computational mesh thereof. The mathematical equations used to characterize the computational modeling embodiment of the present invention are not intended to limit the invention to the mathematical equations used in the model description. Mathematical equations other than those proposed here can be expressed alone or in combination to arrive at the same mathematical results.

A system of partial differential equations (PDEs) can be used to model flow, diffusion, and reaction processes in the 3D system geometry. Time-dependent transport of multiple species (i.e., parent drug(s), metabolite(s), biomarkers, etc.) is characterized by the convective-diffusive-reactive transport equation, given by:

$$\frac{\partial C}{\partial t} + ∇ \cdot ( vC ) = ∇ \cdot D ∇ C + \dot{R}$$

where C is the concentration, t is time, ν is the perfusion velocity, D is the compound-specific diffusion coefficient and \dot{R} is the reaction source term. Fluid transport is determined by simultaneous solving of the Continuity and Momentum Equations as given by:

$$\begin{matrix}Continuity Equation : \frac{\partial }{\partial t} ( ρ ) + ∇ \cdot ( ρ v ) = 0 \\ Momentum Equation : \frac{\partial }{\partial t} ( ρ v ) + ∇ \cdot ( ρ vv ) = - ∇ P + ∇ \cdot ( τ )\end{matrix}$$

where ρ is the fluid density, P is pressure and τ is the shear stress.

Volume conditions, such as density, viscosity and diffusivity, and boundary conditions, such as fluid velocity, pressure, and species concentration, are specifically applied to characterize the system.

For example, in one embodiment of the present invention, a 3D geometry of the microfluidic corneal barrier model was created. In this implementation, the model was built using Computational Biology (CoBi) tools. These tools enabled construction and meshing of the 3D geometry. A structured quadrilateral mesh was applied to the geometry and a grid independence test performed. A multi-physics solver (CoBi) was used to simultaneously solve the system of equations characterizing fluid flow and species transport. The following system operating parameters were applied as boundary conditions: i) fluid flow rates in the TFEp and aqueous humor channels set as constants at the inlets, ii) compound concentration set as a constant at the TFEp inlet and iii) atmospheric pressure was set at all remaining inlets and outlets. Fluid properties of the media were assumed to be identical to those of water and assigned as constant volume conditions. These parameters include density and viscosity. Velocity, pressure, and shear profiles can be generated to verify physiologically relevant shear is achieved in the TFEp and/or AH channels. Species transport of FSS (fluorescein sodium salt) and 4 kD TRITC (tetramethylrhodamine)-dextran can be simulated, and PI calculated for comparison with in vitro data. Compound-specific diffusion coefficients can be calibrated to fit the model to the data. Simulations can be then repeated for cell-seeded devices, where the diffusion coefficient through the porous architecture can be expected to decrease ten-fold. Simulated PIs can then be compared with in vitro data. It is expected that the simulated data can be within the range of the experimental data. This allows for the model to be used to simulate different conditions in the cornea, and can be used for simulating trans-corneal transport.

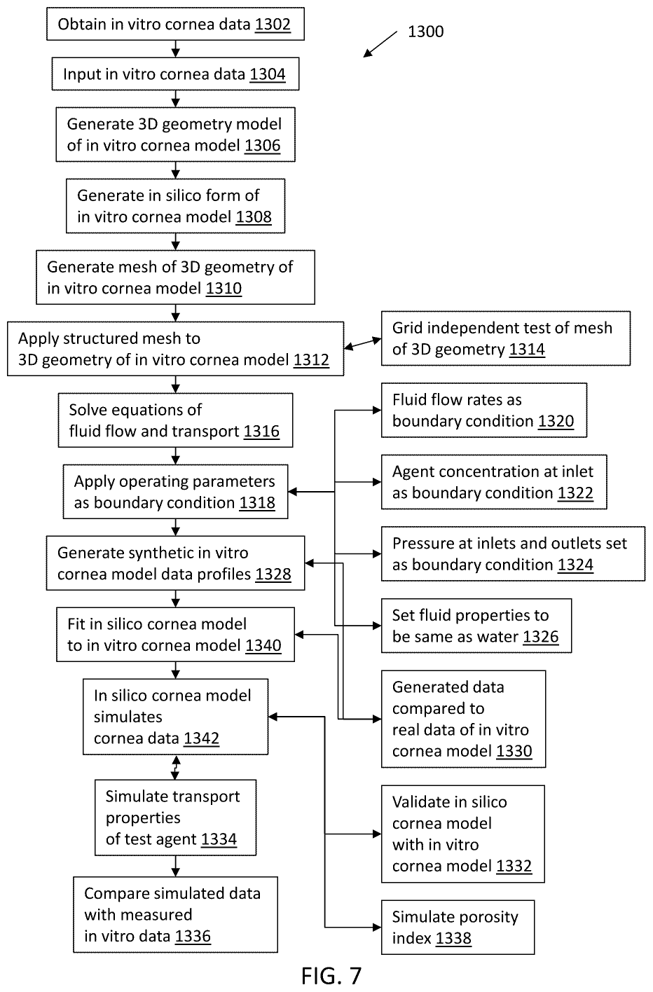

FIG. 7 shows a workflow 1300 for generating an in silico cornea model using data from the in vitro cornea device. The workflow 1300 includes obtaining in vitro cornea data from the in vitro cornea device (Step 1302) and inputting the in vitro cornea data into a computing system (Step 1304). The workflow 1300 also includes computationally generating a 3D geometry model of the in vitro corneal model, and storing the 3D geometry model file on a non-transient storage medium of the computing system (Step 1306). The computational generation of the 3D geometry model of the in vitro corneal model can be performed using computational biology tools to generate construction of an in silico corneal model of the in vitro corneal model (Step 1308) and generating mesh of the 3D geometry of the in vitro corneal model (Step 1310). A structured quadrilateral mesh can be applied to the 3D geometry of the in vitro corneal model (Step 1312). A grid independence test can be performed on the mesh of the 3D geometry (Step 1314). A computational solving module can be used to solve a system of equations that characterize fluid flow and species transport (Step 1316). The computational solving module can include applying operating parameters as boundary conditions for the computational analysis (Step 1318). The boundary conditions can include fluid flow rates in the TFEp and aqueous humor channels set as constants at the inlets (Step 1320). The boundary conditions can also include the concentration of each compound or test agent, which can be set as constant at an inlet to the TFEp chamber of the in vitro cornea device (Step 1322). The boundary conditions can also include the atmospheric pressure being set at all other inlets and outlets of the in vitro cornea device (Step 1324). The computational solving module can set fluid properties to be identical as water and constant volume conditions (Step 1326). The computational solving module can generate synthetic in vitro cornea device data profiles, such as velocity, pressure, and shear (Step 1328). The generated in vitro corneal device data profiles are compared to the real in vitro cornea data (Step 1330), which is used to validate the physical in vitro cornea device and in silico cornea model (Step 1332). The transport properties of a test agent can then be simulated by the in silico cornea model (Step 1334), and these transport properties (e.g., permeability index) can be compared to the corresponding transport properties of the in vitro cornea device (Step 1336). The in silico cornea model can be used to simulate permeability index values, which can be calculated (Step 1338), which can be compared to the permeability index values of the in vitro cornea device data (Step 1334). Compound specific diffusion coefficients can be calibrated to fit the in silico cornea model to the real in vitro cornea device data (Step 1340). This comparison can provide insight as to whether additional transport or elimination mechanisms (e.g., active transport, metabolism) are at play. The in silico cornea model can also be used to optimize operation parameters to mimic physiological conditions in health and disease. The in silico cornea model can be used to perform simulations for the in vitro cornea device, which can be correlated with real cornea data. The in silico cornea can be used to simulate cornea data for one or more test agents (Step 1342).

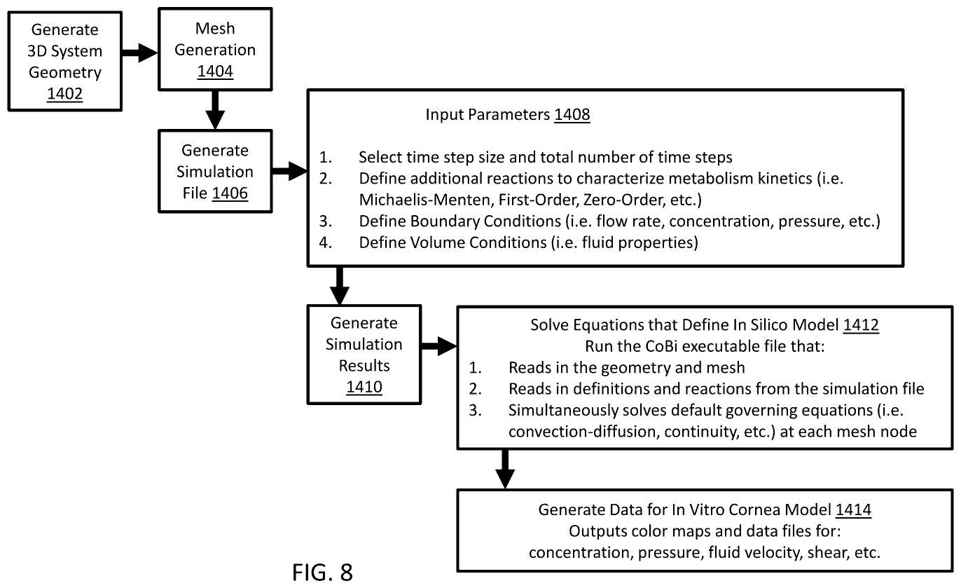

FIG. 8 illustrates a workflow 1400 for providing an in silico cornea model that models the in vitro cornea device. The workflow 1400 generates a 3D system geometry (Step 1402). A mesh is generated for the 3D system geometry (Step 1404). A simulation file for the in silico model is generated for the mesh of the 3D system geometry (Step 1406). Input parameters are input into the system for use with the in silico model (Step 1408). The in silico model generates simulation results for the 3D system geometry (Step 1410). The system solves equations that define the in silico model (Step 1412). Simulation data is generated for the in vitro cornea device (Step 1414). The simulation data can be used to predict the real cornea of a human subject. This allows the in silico cornea model to be used to simulate a real cornea in a human subject. As such, test agents can be simulated in the in silico cornea model in order to obtain data of how the test agent would be taken by a real cornea.

The in silico cornea model can be used to simulate the cornea and assays on the cornea, such as transport of a test agent across the corneal barrier.

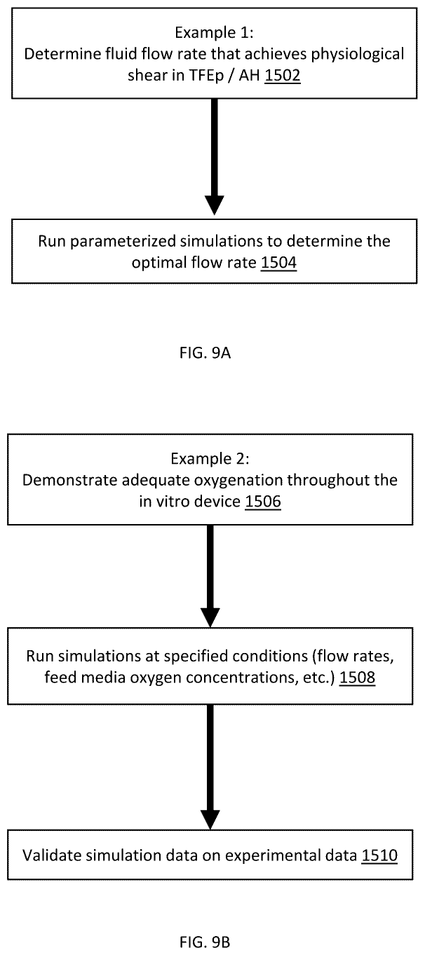

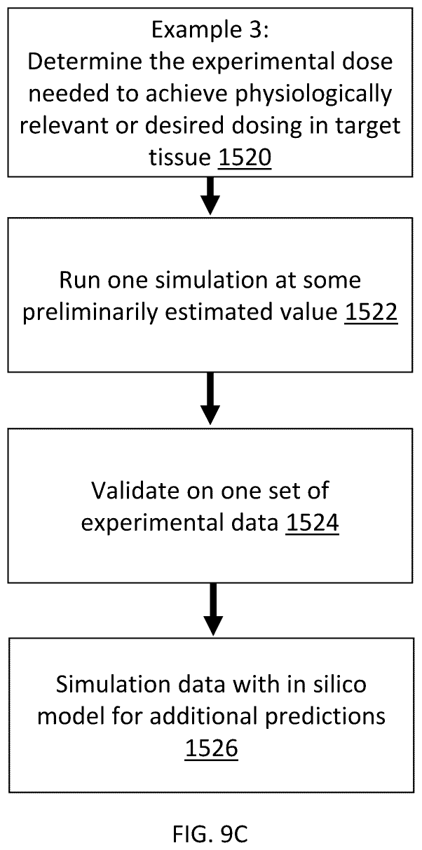

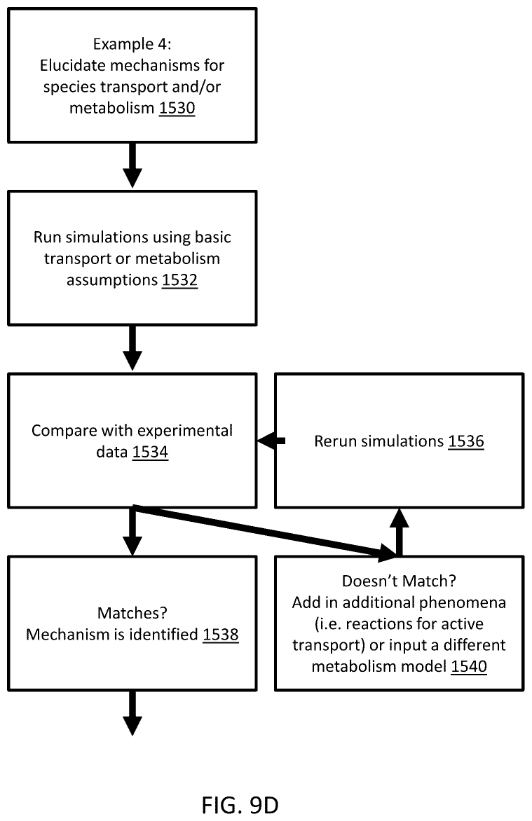

FIG. 9A shows Example 1, which includes determining fluid flow rate that achieves physiological shear in TFEp and/or AH (Step 1502), such as from the in vitro cornea device. Then, the in silico cornea model can be used to run parameterized simulations to determine the optimal flow rate in the in vitro cornea device for specified conditions (e.g., healthy versus diseased) (Step 1504). Then, the in vitro model device can be operated at the optimized flow rate to generate physiologically-relevant in vitro cornea data.

FIG. 9B shows Example 2, which demonstrates adequate oxygenation throughout the in vitro device (Step 1506). The method includes running simulations at specified conditions (e.g., flow rates, test agent concentration, feed media oxygen concentrations, etc.) (Step 1508). Then, the simulation data obtained with the in silico cornea model can be validated with experimental data from the in vitro cornea device (Step 1510).

FIG. 9C shows Example 3, which includes determining the experimental dose of a test agent needed to achieve physiologically relevant or desired dosing in a target tissue, where the dose will go through the corneal barrier (Step 1520). Then, a simulation can be run using the in silico cornea model to obtain simulation data (Step 1522), which can be one simulation that is run at some preliminarily estimated value. The in silico cornea model can be validated on a set of the experimental data from the in vitro corneal model device (Step 1524). The validated in silico model can be used to simulate data for obtaining additional predictions of outcome for test agents and/or test conditions regarding the corneal barrier (Step 1526).

FIG. 9D shows Example 4, which includes a method for improving simulation data of the in silico cornea model. The method can include elucidating mechanisms for species transport and or metabolism of a test agent with the in vitro cornea device (Step 1530). Then, a simulation can be performed using basic transport and/or metabolism assumptions regarding the test agent (Step 1532). The in vitro cornea device data (e.g., experimental data) is compared with the simulation data from the in silico cornea model (Step 1534). When the in vitro cornea device data matches the simulation data, the in silico model is determined to be suitable for modeling a real cornea (Step 1538). When the in vitro cornea device data does not match the simulation data (Step 1540), then the simulation parameters can be modulated, and the simulation can be rerun (Step 1536). This iteration can be performed until a suitable in silico cornea model is obtained that can accurately model a test agent and/or test conditions in a cornea.

### EXAMPLES

### Example In Vitro Cornea Device Embodiments:

In some embodiments, a microfluidic in vitro cornea device is provided for studying a cornea. Such an in vitro cornea device can include: a means of representing a tear flow and/or epithelial (e.g., TFEp), such as a device or system described herein system; a means of representing a corneal stroma barrier; a means of representing an endothelium; and a means of representing an aqueous humor. The device can be configured as an in vitro microfluidic model of a cornea, and can be used to simulate the cornea to obtain data about the behavior of the cornea under certain conditions and in the optional presence of certain test agents. The TFEp system can be configured as a microvasculature structure that is adapted to be coupled to a fluidic network with one or more pumps and optionally one or more media reservoirs. The aqueous humor space can be configured as an eye fluid reservoir (e.g., model thereof), which may optionally be configured to be coupled to a fluidic network with one or more pumps and optionally one or more media reservoirs. The endothelium can be configured as an interfacing region, which may optionally be configured to be coupled to a fluidic network with one or more pumps and optionally one or more media reservoirs. The corneal stroma system can be configured as a tissue structure that is configured to be coupled to a fluidic network with one or more pumps and optionally one or more media reservoirs.

In some embodiments, a microfluidic in vitro cornea device for studying a cornea can include: a TFEp chamber; an stromal chamber adjacent to and porously coupled with the TFEp chamber; an endothelial chamber adjacent to and porously coupled with the stromal chamber; an aqueous humor chamber adjacent to and porously coupled with the endothelial chamber; wherein a first porous wall is positioned between the TFEp chamber and the stromal chamber, a second porous wall is positioned between the stromal chamber and the endothelial chamber, and a third porous wall is positioned between the endothelial chamber and the aqueous humor chamber, which is configured as a microfluidic in vitro model of a cornea.

In some embodiments, the in vitro cornea microfluidic device can include the following in the recited order: the TFEp system; a first porous wall; the stroma tissue space; a second porous wall; the endothelium; a third porous wall; and the aqueous humor circulation system.

In some embodiments, the microfluidic in vitro cornea device can include the following dimensions for the components. The components are recited in the sequential order of a fluid flow or a structural order from a beginning to a terminal end. The TFEp chamber can have a width of about 100 microns, or a range from about 20 microns to about 500 microns, about 50 microns to about 300 microns, about 80 microns to about 200 microns, about 90 microns to about 120 microns. The first porous wall can have a width of about 50 microns, or a range from about 20 microns to about 100 microns, about 25 microns to about 90 microns, about 30 microns to about 85 microns, or about 40 microns to about 60 microns. The stromal chamber can have a width of about 450 microns, or a range from about 100 microns to about 1000 microns, from about 200 microns to about 850 microns, about 300 microns to about 600 microns, or about 400 microns to about 500 microns. The second porous wall can have a width of about 50 microns, or a range from about 20 microns to about 100 microns, from about 35 microns to about 90 microns, from about 40 microns, to about 75 microns, or about 45 microns to about 60 microns. The endothelial chamber can have a width of about 100 microns, or a range from about 10 microns to about 200 microns, about 40 microns to about 160 microns, about 60 microns to about 120 microns, or about 90 microns to about 110 microns. The third porous wall can have a width of about 50 microns, or a range from about 20 microns to about 100 microns, from about 35 microns to about 90 microns, from about 40 microns, to about 75 microns, or about 45 microns to about 60 microns. The aqueous humor chamber can have a width of about 2000 microns, or a range from about 100 microns to about 5000 microns. The height of the device, or the height of any of the foregoing chambers or features can vary from about 10 microns to about 1000 microns, from about 50 microns to about 500 microns, or about 100 microns to about 200 microns, or about 150 microns. These values may be varied, such as +/−1%, 5%, 10%, 25%, 50%, 75%, or 100% thereof.

In some embodiments, the in vitro cornea microfluidic device can be configured with the following dimensions of the components, which are recited in an order or arrangement from a beginning to a terminal end. The TFEp system can have an arc length of about 15.5 mm, or a range from about 5 mm to about 50 mm, from about 8 mm to about 30 mm, or about 10 mm to about 20 mm. The first porous wall can have an arc length of about 15.2 mm, or a range from about 5 mm to about 50 mm, about 7 mm to 30 mm, or about 10 mm to about 600 mm. The stroma tissue space can have an arc length of about 14.8 mm, or a range from about 5 mm to about 50 mm, about 8 mm to about 30 mm, or about 10 mm to about 20 mm. The second porous wall can have an arc length of about 14.3 mm, or a range from about 5 mm to about 50 mm, about 7 mm to 30 mm, or about 10 mm to about 20 mm. The endothelium can have an arc length of about 14.1 mm or a range from about 5 mm to about 50 mm, about 8 mm to about 30 mm, or about 10 mm to about 20 mm. The third porous wall can have an arc length of about 14.0 mm microns or a range from about 5 mm to about 50 mm, about 8 mm to about 30 mm, or about 10 mm to about 20 mm. The aqueous humor circulation system can have a length (e.g., of base of dome) of about 12.5 mm, or a range from about 5 mm to about 50 mm, about 8 mm to about 30 mm, or about 10 mm to about 20 mm. In some aspects, these values can be modified, such as by +/−1%, 5%, 10%, 25%, 50%, 75%, or 100% thereof. These lengths can be seen to extend between the dashed lines of the assay region 112 of FIG. 1A.

In some embodiments, each porous wall can include pore channels therethrough that are 5 microns wide that are spaced 50 microns apart. The width of each pore channel may range from about 1 micron to about 10 microns, about 2 microns to about 8 microns, or about 3 microns to about 7 microns. The heights of each pore channel can ranges from about 6 microns to about 10 micron, or about 8 microns. The spacing of the channels can be from about 5 microns to about 100 microns, about 10 microns to about 90 microns, or about 25 microns to about 75 microns. These values may be modified, such as by +/−1%, 5%, 10%, 25%, 50%, 75%, or 100% thereof. In some aspects, the device can include a porous wall between the stroma tissue space and the endothelium. In some aspects, the device can include a porous wall between the endothelium and the aqueous humor circulation system.

In some embodiments, the aqueous humor chamber includes a plurality of support posts. The support posts can be dimensioned and arranged so as to be configured to stop collapse under physiological fluidic flow and pressure. The support posts can be used to maintain mechanical stability of the in vitro device. The device can also include a lid, wherein the lid is supported by the support posts.

In some embodiments, the in vitro cornea device can include a viewing window into at least one of the TFEp chamber, stroma tissue chamber, endothelium, or aqueous humor chamber. However, each of these systems or chambers can be configured for optical viewing. These systems or chambers can have a clear lid or top, or any other surface, to allow for optical viewing of the content thereof. This can allow for labeling and other colorimetric techniques for monitoring the cultures as well as flow of components and test agents throughout the system.

In some embodiments, an assay chip can be configured with the in vitro cornea device. The assay chip can include a substrate; and a plurality of the microfluidic in vitro cornea devices in or on the substrate. The plurality of microfluidic in vitro models can be in parallel or sequential, or combinations thereof.

In some embodiments, a microfluidic in vitro cornea system can include: the microfluidic in vitro cornea device of one of the embodiments; and at least one pump configured for moving fluid through the microfluidic in vitro cornea device.

In some embodiments, a microfluidic in vitro cornea system can include: the microfluidic in vitro cornea device of one of the embodiments; at least one camera device configured to be positioned to image at least one of the TFEp chamber, stromal chamber, endothelial chamber, or aqueous humor chamber; and a computing system operably coupled with the at least one camera device to receive image data. In some aspects, the computing system is configured to obtain data from the at least one camera device and determine at least one trans-cornea transport barrier property of the microfluidic in vitro cornea device or at least one trans-cornea transport property of a test agent, wherein the trans-cornea transport barrier property is a measurement of inhibition of transport of an agent across the corneal barrier and the trans-cornea transport property of a test agent is a measurement of traversal of the test agent across the corneal barrier. In some aspects, the computing system includes one or more computer-readable media storing instructions that when executed cause operations that determine the at least one trans-cornea transport barrier property of the microfluidic in vitro cornea device or the at least one trans-cornea transport property of a test agent.

In some embodiments, the in vitro cornea device can be used in a method of studying the cornea. The method can include providing the in vitro cornea device of one of the embodiments. For example, the device includes TFEp having epithelial cells in the TFEp system, stoma cells in the stromal chamber, endothelial cells in the endothelial chamber, and fluid in the aqueous humor chamber. Optionally, there are endothelial cells in the aqueous humor circulation system. The device can be used for determining a condition of the in vitro cornea device at a first time point. The condition can be any physiological condition of the cells, fluid flow, test analyte, or other feature. Then, the method can include determining the condition of the in vitro cornea device at a subsequent time point. That is, the same feature can be measured at a later time, such as after exposure to a certain condition or test analyte. Then, a computing system can be used for determining a change in the condition of the in vitro cornea device.

In some embodiments, the method of studying the cornea can include: measuring a barrier function property of the corneal barrier; imaging the TFEp chamber, stromal chamber, endothelial chamber, or aqueous humor chamber through a viewing window of the device; viewing images in real time of the TFEp chamber, stromal chamber, endothelial chamber, or aqueous humor chamber through a display screen of a computing system; or measuring transport across the corneal barrier of at least one of nutrients, xenobiotics, small molecules, lipids, liposomes, polymers, particles, toxins, and antibodies from the images.

In some embodiments, the method of studying the cornea can include: providing the microfluidic in vitro cornea device of one of the embodiments having epithelial cells in the TFEp chamber, corneal stroma cells in the stromal chamber, endothelial cells in the endothelial chamber, and fluid with or without cells in the aqueous humor chamber; providing a test agent to an input chamber selected from the TFEp chamber or aqueous humor chamber; and monitoring trans-corneal transport of the test agent.

In some embodiments, a method of studying transport of the test agents can include at least one of: determining an amount of test agent crossing the corneal barrier and comparing the amount of test agent that crossed the corneal barrier (e.g., stroma, endothelial and aqueous humor chambers) with the administered amount of the test agent introduced into the microfluidic in vitro cornea device; sampling the aqueous humor chamber for the test agent and quantifying the transport of the test agent across the corneal barrier into the aqueous humor chamber; or sampling the TFEp chamber for the test agent and quantifying the transport of the test agent across the corneal barrier into the TFEp chamber.

In some embodiments, the methods can include evaluating corneal barrier function of the corneal barrier by: injecting a plurality of different test agents having a plurality of different sizes into the TFEp chamber; imaging the in vitro cornea device; analyzing images of the in vitro cornea device to identify the plurality of different test agents; and determining a size of test agent or size range of test agent of the plurality of test agents located in the TFEp chamber, stromal chamber, endothelial chamber, and/or aqueous humor chamber.

In some embodiments, the methods can include determining at least one of: a size of test agent or size range of test agents capable of transporting from the TFEp chamber across the stromal chamber and endothelial chamber into the aqueous humor chamber; a lipophilicity of test agent or lipophilicity range of test agents capable of transporting from the TFEp chamber across the stromal chamber and endothelial chamber into the aqueous humor chamber; or a physiological charge of test agent or charge range capable of transporting the TFEp chamber across the stromal chamber and endothelial chamber into the aqueous humor chamber.

In some embodiments, the methods can include evaluating permeability of the in vitro cornea device by: injecting one or more test agents into the TFEp chamber; imaging the microfluidic in vitro cornea device; analyzing images of the microfluidic in vitro cornea device to identify locations of the test agent at defined time points, and optionally determine amounts of each test agent in each chamber at the time points; and determining a permeability of the in vitro cornea device for the one or more test agents.

In some embodiments, the methods can include determining a permeability index as a ratio of optical intensity measurements of the TFEp chamber with the aqueous humor chamber.

In some embodiments, the methods can include evaluating whether the test agent modifies permeability or structural integrity or morphology of the corneal epithelial cells in the TFEp, fibroblasts in the stromal chamber and/or endothelial cells in the endothelial chamber by: determining an initial value of a first property of the corneal epithelial, stromal and/or endothelial chamber; introducing the test agent into the microfluidic in vitro cornea device; determining a subsequent value of the first property of the corneal epithelial, stromal and/or endothelial chamber; and determining a difference between the initial value and the subsequent value of the first property of the corneal epithelial, stromal and/or endothelial chamber. The value can be related to one or more of structural integrity or morphology of the corneal epithelial, stromal and/or endothelial chamber or other feature, as well as features of the test agent. Accordingly, reference to a chamber refers to the cells, cell culture, or tissue culture therein.

In some embodiments, the methods can include determining a health consequence of the test agent modulating the corneal stroma barrier chamber and/or endothelial chamber by correlating the difference between the initial value and the subsequent value and a phenotypic state, which phenotypic state may or may not be a disease state or disorder state.

In some embodiments, the method of studying the cornea can include obtaining in vitro transport data, and converting the in vitro transport data into synthetic in vivo transport data with a computational cornea model. Here, the system can use the actual data from the in vitro device, such as over a number of runs, and then create synthetic in vivo transport data therefrom. The conversion can use a computational model that correlates the in vitro data to in vivo transport data.

In some embodiments, the method of studying the cornea can include measuring a barrier function property of the corneal barrier. The corneal barrier can include the endothelial cell culture, first porous wall, stroma cell culture, second porous wall, endothelial culture, such as for transfer from the TFEp fluid to the aqueous humor fluid. Here, at least one test analyte can be used with the device to test the transport of the test analyte across the corneal stroma barrier and. Then, the data thereof can be used for determining a barrier function property based on the measured data.

In some embodiments, the method of studying the cornea can include optically viewing, such as with human eyes, camera, video or other recorder the different components of the device. As such, the following components can be configured for visual monitoring, and may include camera devices to record the TFEp chamber, stromal chamber, endothelial chamber, or aqueous humor chamber through a viewing window of the device.

In some embodiments, the method of studying the cornea can include performing in real-time, quantitative imaging of: stroma fibroblast interactions; the fibroblast response to drug exposure; and biological secretions. This monitoring can be done with a camera system and visualization of the contents of each component. Also, dyes and other markers can be used to track different features of the stroma cell activity and biological secretions.

The method of studying the cornea can include validating the in vitro cornea device on TFEp to aqueous humor transport of selected nutrients, xenobiotics, lipids, toxins and antibodies. Here, the data obtained from the in vitro model can be compared to known in vivo data for these types of test analytes. As such, these test analytes may be used as controls in the different assays.

In some embodiments, the method of studying the cornea can include performing assays for one of the following on any of the compartments or components of the in vitro device, such as viability, and phenotypic expression assays. The data from these assays can be used to optimize and characterize the in vitro model.

In some embodiments, the in vitro cornea device can be used in a method of studying transport of test agents across the cornea. The in vitro model can be used to simulate the in vivo cornea. The method can include providing the in vitro cornea device of one of the embodiments and providing a test agent to the TFEp system. Then, various techniques can be used for monitoring the test agent in the aqueous humor circulation system. The amount of test agent from the TFEp system or aqueous humor system provides information for the transport of that test agent, which simulates the in vivo transport.

In some embodiments, the amount of the test agent that crosses the corneal barrier can be determined from the amount that makes it from the TFEp system into the aqueous humor circulation system. The data can be analyzed by comparing the amount of test agent in the aqueous humor circulation system with the administered amount of the test agent introduced into the TFEp system. The amount that passes through the model is indicative of the function of the cornea and ability of the test agent to also transport through the cornea. In some aspects, the method of studying transport of test agents can include sampling the aqueous humor circulation system for the test agent, and quantifying the transport of the test agent into the aqueous humor circulation system. The configuration of the in vitro cornea device with optical viewing windows allows for performing a real-time study of cellular behavior, drug delivery and efficacy with the in vitro cornea device.

The different components of the in vitro cornea device can be evaluated. The method of studying transport of test agents can include determining a TFEp (e.g., epithelial) contribution to transport resistance in the in vitro cornea device. Additionally or alternatively, the method can include evaluating corneal barrier function of the in vitro cornea device. For example, the protocol for evaluating corneal barrier function of the in vitro cornea device can be performed by: injecting a plurality of different test agents having a plurality of different sizes into the TFEp system; imaging the in vitro cornea device; analyzing images of the in vitro cornea device to identify the test agent; and determining a size of test agent or size range of test agent located in the TFEp system, stroma tissue space, endothelium, and/or aqueous humor circulation system. Different sizes can include different coloring or other markers; however, the size of the test agents can be monitored by the known size and optical tracking of the particles through the flow path of the in vitro cornea device. Also, the protocol can include determining a size of test agent or size range of test agent capable of transporting from the TFEp system to the aqueous humor circulation system.

In some embodiments, the method of studying transport of test agents can include determining a lipophilicity of test agent or lipophilicity range of test agent capable of transporting from the TFEp system to the aqueous humor circulation system. As such, a panel of different agents with a gradient of lipophilicity can be used and monitored in order to create a lipophilicity profile for test agents, which can provide a lipophilicity range of the in vitro device. The amount of lipophilicity of a certain substance type can be assessed to determine whether such lipophilicity modulates the transport thereof or other test agents in the in vitro cornea device.

In some embodiments, the method of studying transport of test agents can include determining a physiological charge of test agent or range thereof capable of transporting from the TFEp system to the aqueous humor circulation system. Accordingly, the different charges of different test agents can be monitored across a panel for transport in the in vitro cornea device. The pH may also be varied to monitor the modulation that charges can have on the transport phenomena.

In some embodiments, the method of studying transport of test agents can include evaluating permeability of the in vitro cornea device. The permeability may compromise the transport data that can be determined to make sure the data is physiologically relevant to in vivo cornea parameters. The permeability can be assessed by: injecting test agent into the TFEp system; imaging the in vitro cornea device; analyzing images of the in vitro cornea device to identify locations of the test agent and optionally determine amounts of test agent in each location; and determining a permeability of the in vitro cornea device for the test agent. The test agent may or may not be labeled with a visible label, such as fluorescent label. The method can also include evaluating permeability of the in vitro cornea device by: injecting test agent into the TFEp system; imaging the aqueous humor circulation system; analyzing images of the aqueous humor circulation system to identify the test agent; and determining a permeability of the in vitro cornea device for the test agent. The evaluation of permeability of the in vitro cornea device can also be performed by: injecting test agent into the TFEp system; imaging the TFEp system; analyzing images of the TFEp system to identify the test agent; and determining a permeability of the in vitro cornea device for the test agent. Also, the permeability of the in vitro cornea device can be evaluated by: injecting test agent into the TFEp system; imaging the stroma tissue space; analyzing images of the stromal chamber to identify the test agent; and determining a permeability of the in vitro cornea device for the test agent. In another aspects, the protocol for evaluating permeability of the in vitro cornea device can be performed by: injecting test agent into the TFEp system; imaging the endothelial chamber; analyzing images of the endothelial chamber to identify the test agent; and determining a permeability of the in vitro cornea device for the test agent. These studies may be used alone or in any combination for monitoring the permeability.

In some embodiments, the method of studying transport of test agents can include determining a permeability index. The permeability index can be defined as a ratio of a sum of optical intensity measurements of one or more region of interests (ROIs) in the stroma culture or the endothelium in comparison with the fluid channels of the TFEp and aqueous humor circulation systems. This ratio between the optical intensity measurements of the TFEp and aqueous humor circulation systems can be used to define the permeability. Accordingly, the method can include: determining a permeability index for the TFEp system relative to the endothelium; determining a permeability index for the aqueous humor circulation system relative to the endothelium; and determining a difference in permeability index for the TFEp system compared to the aqueous humor circulation system. The permeability index of each chamber can be assessed, as well as the permeability across multiple chambers or the entire device from the TFEp to the aqueous humor.

In some embodiments, the methods can also include evaluating whether an agent modifies permeability of the in vitro model, or the TFEp system, stromal chamber, endothelial chamber, and/or aqueous humor circulation system. As such, a test agent can be used with other test substances (e.g., positive or negative controls) to see if there is a modification of the permeability of the test substance. These types of agents may be used in treatments to either increase or decrease the permeability of the cornea. For example, the protocol can include evaluating whether a test agent modulates structural integrity of the endothelium by: determining an initial value of a first property of the endothelium; introducing the test agent into the in vitro microfluidic model; determining a subsequent value of the first property of endothelium; and determining a difference between the initial value and the subsequent value. The protocols can also include determining a health consequence of the test agent modulating structural integrity by correlating the difference between the initial value and the subsequent value and a phenotypic state, which may or may not be a disease state or disorder state. An example of monitoring structural changes can include determining a morphological change to the endothelium after treatment with the test agent. This can be repeated for each chamber, and can be used for studying all of the chambers at the same time.

### Example In Silico Cornea Model Embodiments:

In some embodiments, a method of computationally modeling a cornea can include: generating a 3D geometry computer model of an in vitro cornea device; generating a mesh of the 3D geometry computer model; generating a computer model of the in vitro cornea device with the mesh; and processing the computer model through an solver module to obtain an in silico cornea model.

In some embodiments, a method of computationally modeling a cornea can be performed. The computationally modeled cornea can be used for simulating an in silico cornea to generate data to represent an in vivo cornea. The computationally modeled cornea can be obtained by obtaining data from the in vitro cornea device of one of the embodiments, wherein the in vitro model device is a physical model of a cornea. Then, the computing system can be used for creating an in silico computational model of the in vitro model device. Then, the computing system can generate a physiology-based pharmacokinetic (PBPK) model of the cornea. The computationally modeled cornea can be used for predicting transport efficiency of the physical in vitro cornea device. Accordingly, certain test agents can be used for the in vitro model to obtain a corresponding in silico model of the cornea. For example, the computationally modeled cornea can be created by obtaining in vitro transport data, and converting the in vitro transport data into synthetic in vivo transport data with the corneal PBPK model.

The in silico cornea can be obtained as described and used in a number of different types of studies for the cornea. In one example, the in silico cornea model can be used for determining how some properties (e.g., one or more of protein binding, ionization, lipophilicity and molecular weight) impact trans-corneal transport. Accordingly, relevant data of the test agent can be obtained, such as the foregoing, and the data can be input into the computing system.

In some embodiments, the in silico corneal model can be used for computationally modeling the cornea by corelating aqueous humor exposure to the test compound and toxicity to the eye.

In some embodiments, the computationally modeled cornea can be used for modeling an endothelium contribution to transport resistance.

In some embodiments, the computationally modeled cornea can be used for evaluating mechanisms of trans-corneal transport.

In some embodiments, the computationally modeled cornea can be used for performing a sensitivity analysis to predict which factors are likely to be rate limiting for transfer and to identify factors that lead to corneal dysfunction and increased aqueous humor concentration or eye toxicity.

In some embodiments, the computationally modeled cornea can be configured as a physiology-based pharmacokinetic (PBPK) model with a high-resolution three dimensional model of the cornea.

In some embodiments, the computationally modeled cornea can be configured as the corneal PBPK model for a corneal transport model based on the complex structure and physiology of the cornea.

In some embodiments, the computationally modeled cornea can be configured as a corneal PBPK model that can be used for mechanistic modeling of trans-corneal transport, considering paracellular, transcellular, transporter mediated transport, and metabolism kinetics.

In some embodiments, the computationally modeled cornea can be configured as the corneal PBPK model, which is extrapolated from the in silico cornea model that is based on the in vitro cornea device.

In some embodiments, the method of computationally modeling the cornea can include importing data into a multi-physics solver module, which: simultaneously solves a system of equations of fluid flow and species transport. In some aspects, the in silico cornea model is dimensioned as the in vitro microfluidic cornea model. To have variability in cornea size, the in silico cornea model or corneal PBPK model is operable across a range of mesh sizes.

In some embodiments, the method of computationally modeling the cornea can include boundary conditions. For example, the in silico cornea model includes boundary conditions as follows: i) fluid flow rates in the TFEp and aqueous humor channels set as constants at the inlets, ii) fluorescent compound concentration set as a constant at the TFEp inlet, and iii) atmospheric pressure was set at all remaining inlets and outlets. The operation can include the fluid properties of the media being assumed to be identical to water and assigned as constant volume conditions, wherein the fluid properties optionally include density and viscosity.

In some embodiments, a method of computationally modeling the cornea can include: simulating the in vitro cornea device with the in silico cornea model; comparing simulation data of the in silico cornea model with real data from the in vitro cornea device; and iterating simulation parameters until simulation data matches the real data.

In some embodiments, a method of computationally modeling the cornea can include calibrating diffusion coefficients for one or more test agent to fit the simulation data of the in silico cornea model or the corneal PBPK model with the real data of the in vitro cornea device.

In some embodiments, a method of computationally modeling the cornea can include using a machine learning platform having a deep neural network for optimizing the in silico cornea model to simulate the in vitro cornea device. Some aspects can include using a machine learning platform having a deep neural network for optimizing the in silico cornea model or corneal PBPK model with data from the in vitro cornea device or real corneal data to simulate an in vivo cornea. Additionally, machine learning could be used to make predictions about trans-corneal transport of other compounds based on similarity of a test agent's (e.g., compound that is known, previously tested, etc.) physicochemical properties and permeability data, such as from previously tested agents.

In some embodiments, the method of computationally modeling the cornea can include configuring the in silico cornea model or corneal PBPK model for analyzing flow-induced shear and species transport.

In some embodiments, a method of computationally modeling the cornea can include using simulation data from the in silico cornea model for obtaining the corneal PBPK model.

In some embodiments, a method of computationally modeling the cornea can be used for obtaining corneal PBPK model predictions for time-dependent concentrations of molecules in aqueous humor circulation are compared with experimental data from the in vitro cornea device and with real data (e.g., literature).

In some embodiments, a method of computationally modeling the cornea includes the in silico cornea model considering convective-diffusive transport in calculations and determinations of transport properties and value of various types of test agents. In some aspects, the in silico cornea model considers convective-diffusive transport, transporter mediated transport, metabolism, and combinations thereof.

In some embodiments, a method of computationally modeling the cornea includes a corneal PBPK model being configured for obtaining data regarding specific transporters, metabolizing enzymes, and other parameters.

In some embodiments, a method of computationally modeling the cornea includes the corneal PBPK model being configured for predicting factors that are rate limiting steps for transfer of an agent across the stromal tissue and/or endothelium.

In some embodiments, a method of computationally modeling the cornea can include the corneal PBPK model being configured for modeling passive corneal diffusion by Fick's law.

In some embodiments, a method of computationally modeling the cornea can include the corneal PBPK model being configured for modeling the cornea with distribution differences related to partitioning (e.g., based on LogP), size exclusion (e.g., MW) and exclusion of ionized compounds.

In some embodiments, a hybrid cornea model system can include a physical in vitro cornea device of one of the embodiments, and an in silico cornea model of one of the embodiments, which is generated based on data of the in vitro cornea device. In some embodiments, a computational cornea model correlates in vitro data from the physical cornea model with in vivo data, and provides synthetic in vivo data for a model cornea.

In some embodiments, a corneal PBPK model is prepared from the in silico cornea model with in vivo data. The in vivo data can be obtained from experiment, literature, or other. The in vivo data is used with the in silico cornea model to create the corneal PBPK model.

In some embodiments, a method of simulating a cornea can include: providing the corneal PBPK model; simulating a condition in a cornea with the corneal PBPK model; and determining an outcome of the condition in a real cornea based on the outcome of the corneal PBPK model. The condition can be any type of physical condition, or compositional condition. Different test agents can also be used as test agent condition, which can be used for screening. In some aspects, the condition is a test agent, and the outcome is transport of the test agent across the cornea into an eye.

In some embodiments, a method of computationally modeling a cornea can include: generating a 3D geometry computer model of an in vitro cornea device; generating a mesh of the 3D geometry computer model; generating a computer model of the in vitro cornea device with the mesh; and processing the computer model through an solver module to obtain an in silico cornea model.

In some embodiments, a method of computationally modeling a cornea can include: obtaining data from an in vitro cornea device, wherein the in vitro cornea device is a physical model of a cornea; creating a computational model of the in vitro model device; and generating a physiology-based pharmacokinetic (PBPK) model of the cornea as the in silico cornea model.

The method of computationally modeling a cornea can include: applying a structured quadrilateral mesh to the 3D geometry computer model; performing a grid independence test; and determining the mesh for the 3D geometry computer model to be valid for the in vitro cornea device.

In some embodiments, a method of computationally modeling a cornea can include solving equations of fluid flow and species transport of the 3D geometry computer model.

In some embodiments, a method of computationally modeling a cornea can include setting operating parameters of the in silico cornea model, which operating parameters are applied as boundary conditions.

In some embodiments, the method of computationally modeling a cornea can include an in vitro cornea device that comprises: a TFEp chamber; a stromal chamber adjacent to and porously coupled with the TFEp chamber; an endothelial chamber adjacent to and porously coupled with the stroma tissue space; an aqueous humor chamber adjacent to and porously coupled with the endothelial chamber; wherein: a first porous wall is positioned between the TFEp chamber and the stromal chamber, a second porous wall is positioned between the stroma chamber and the endothelial chamber, and a third porous wall is positioned between the corneal barrier and the aqueous humor chamber, the TFEp chamber includes corneal epithelial cells; the stromal chamber includes fibroblasts; the endothelial chamber includes endothelial cells; and the aqueous humor chamber includes a fluid to mimic the aqueous humor; which is configured as a microfluidic in vitro model of a cornea.

In some embodiments, a method of computationally modeling a cornea can include the operating parameters as follows: setting fluid flow rates in the TFEp chamber; setting fluid flow rates in the aqueous humor chamber; setting a test agent concentration as a constant at an inlet of the TFEp chamber; and setting atmospheric pressure at all remaining inlet and outlets of the in vitro cornea device.

In some embodiments, the method of computationally modelling a cornea can include setting fluid properties to be identical with water as constant volume conditions.

In some embodiments, a method of computationally modelling a cornea can include at least one of: generating velocity, pressure, and/or shear profiles for one or more flow rates in the in vitro cornea device; generating velocity, pressure, and/or shear profiles with the in silico cornea model; and validating whether or not physiological relevant sheer is achieved in the in vitro cornea device and/or the in silico cornea model.

In some embodiments, a method of computationally modelling a cornea can include at least one of: generating a porosity index of one or more test agents for one or more flow rates in the in vitro cornea device; generating a porosity index of the one or more test agents with the in silico cornea model; and validating the porosity index of the one or more test agents with the in vitro cornea device and/or the in silico cornea model.

In some embodiments, a method of computationally modeling a cornea can include fitting the in silico cornea model with the in vitro cornea device data.

In some embodiments, a method of computationally modeling a cornea can include: inputting input parameters for one or more conditions and/or one or more test agents into the in silico cornea model; and generating simulated cornea data for the one or more conditions and/or one or more test agents.

In some embodiments, a method of computationally modeling a cornea can include generating a simulation file by: selecting time step size and total number of time steps; defining additional reactions to characterize metabolism kinetics; and defining boundary conditions; and defining volume conditions.

In some embodiments, a method of computationally modeling a cornea can include generating simulation results of the in silico cornea model.

In some embodiments, a method of computationally modeling a cornea can include solving equations that define the in silico cornea model by: reading geometry and mesh data; reading definitions and reactions from the simulation file; and simultaneously solving the equations at east mesh node.

In some embodiments, a method of computationally modeling a cornea can include: generating data for in vitro cornea device; and outputting data files for concentration, pressure, fluid velocity, and shear rate for one or more test agents at one or more locations in the in vitro cornea device. A method of computationally modeling a cornea can include outputting simulated cornea data.

In some embodiments, a method of simulating a cornea can include: providing an in silico cornea model that is based on an in vitro cornea device; inputting test parameters for one of more test agents and/or one or more test conditions into the in silico cornea model; and generating simulated cornea data that simulates the one or more test agents and/or one or more test conditions in a real cornea.

In some embodiments, a method of simulating a cornea can include: determining an experimental dose of a test agent to achieve a target dosing amount to cross a corneal barrier; running at least one simulation at a preliminary estimated value for the test agent; validating the simulation with experimental data from the in vitro cornea device; and simulating cornea data with the validated in silico cornea model.

In some embodiments, a method of simulating a cornea can include: elucidating one or more mechanisms for transport of a test agent and/or metabolism of the test agent in a cornea; running a simulation for the test agent with the in silico cornea model using one or more basic transport definitions and/or one or more basic metabolism definitions to obtain simulated cornea data; comparing the simulated cornea data with experimental data from the in vitro cornea device; and determining whether or not the simulated cornea data matches the experimental data.

In some embodiments, a method of simulating a cornea can include determining that the simulated data matches the experimental data and providing a report with the simulated data.

In some embodiments, a method of simulating a cornea can include determining that the simulated data does not match the experimental data, and then: changing at least one input, operating parameter, or boundary condition; and rerunning the simulation for the test agent.

In some embodiments, a method of simulating a cornea can include: obtaining in vitro transport data; and converting the in vitro transport data into synthetic in vivo transport data with the in silico cornea model.

In some embodiments, a method of simulating a cornea can include one or more of: determining one or more of protein binding, ionization, lipophilicity and molecular weight of one or more test agents, and inputting the same into the computing system; correlating eye exposure to the test compound and toxicity to the eye or entire human; modeling a stromal and/or endothelium contribution to transport resistance; evaluating mechanisms of trans-corneal transport; or performing a sensitivity analysis to predict which factors are likely to be rate limiting for transfer and to identify factors that lead to corneal dysfunction and increased eye and whole body or downstream toxicity.

In some embodiments, a method of simulating a cornea, wherein the in silico cornea model is used for mechanistic modeling of trans-corneal transport, paracellular, transcellular, transporter mediated, and metabolism mechanisms.

In some embodiments, a method of simulating a cornea using the in silico cornea model includes boundary conditions as follows: i) fluid flow rates in TFEp and aqueous humor chambers set as constants at the inlets, ii) fluorescent compound concentration set as a constant at a TFEp inlet, and iii) atmospheric pressure is set at all remaining inlets and outlets of the in vitro cornea device, wherein fluid properties of the media are assumed to be identical to water and assigned as constant volume conditions, wherein the fluid properties optionally include density and viscosity.

In some embodiments, a method of simulating a cornea can include: simulating the in vitro cornea device with the in silico cornea model; comparing simulation data of the simulation with real data from the in vitro cornea device; and iterating the simulation until simulation data matches the real data.

In some embodiments, a method of simulating a cornea can include calibrating diffusion coefficients for one or more test agent to fit the simulation data of the in silico cornea model or the corneal PBPK model with the real data of the in vitro cornea device.

In some embodiments, a method of simulating a cornea with the in silico cornea model can include: considering convective-diffusive transport, transporter mediated transport, metabolism, and combinations thereof; and/or obtaining data regarding specific transporters, metabolizing enzymes, and other parameters; and/or predicting factors that are rate limiting steps for transfer of an agent across the stroma or endothelium, with or without the endothelium The in silico corneal model can model passive corneal diffusion by Fick's law; or can model with distribution differences related to partitioning (based on LogP), size exclusion (MW) and exclusion of ionized compounds.

In some embodiments, a hybrid cornea model system includes: a physical in vitro cornea device; and an in silico cornea model as obtained in one of the embodiments. The in silico cornea model can be on a computing system or on one or more non-transient memory devices with computer-executable instructions to perform the methods recited herein.

### EXAMPLES

### Microfabrication of Novel Microfluidic Corneal Barrier Platform.

A physiologically relevant corneal platform can be designed and developed using microfabrication methodologies. The microfabricated platform can be tested for structural and fluidic integrity using fluorescent molecules and particles.

Corneal Barrier Co-Culture in the Microfluidic Platform.

Using a native corneal collagen-based extracellular matrix, a co-culture comprised of epithelial cells and fibroblasts can be generated. The TFEp can include epithelial cells. Conditions can be optimized for media composition, seeding densities and flow pattern (pulsatile vs. constant) in combination with morphological and functional evaluation, including viability, tight junction formation (e.g., permeability of small molecules) and stroma tissue formation. Finally, production of cornea-specific phenotypic markers (e.g., collagen 1, mucin 1, CK3, etc.) expressed at physiologically relevant levels can be evaluated to validate corneal phenotype

### Barrier Function in the Human Corneal Co-Culture Model.

Corneal barrier function can be demonstrated using positive control compounds that are known to readily cross the barrier (e.g., fluorescein) and negative control compounds that are known to not readily cross the barrier (e.g., 10 kD FITC-dextran). Experimental trans-corneal transport data can be compared with explant and in vivo data from literature. Toxicological effects on barrier integrity can be evaluated (e.g., tight junction formation, permeability).

### Computational Model of the Corneal Barrier Platform.

Development of an in silico model at the experimental microscale level allows creation of an interconnected physiologically-based pharmacokinetic (PBPK) corneal in silico model for predicting aqueous humor, eye, or whole body exposure and toxicity. The fully developed in silico model will include a high-resolution 3D model of the cornea which connects TFEp and aqueous humor PBPK models. In silico trans-corneal drug transport can be characterized by results from the in vitro experiments (e.g., diffusion rate and permeability). As such, prediction of trans-corneal transport of one or more test agents by in silico simulation in the in silico barrier platform can be used to predict outcomes of real cornea. Specifically, using CoBi modeling tools, the in silico cornea model can characterize trans-corneal drug transport based on physicochemical properties (e.g., molecular weight, lipophilicity, ionized state), tissue properties (e.g., pH, thickness), and physics (e.g., convection-diffusion equation). Model simulations of known small molecules will be performed and validated.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

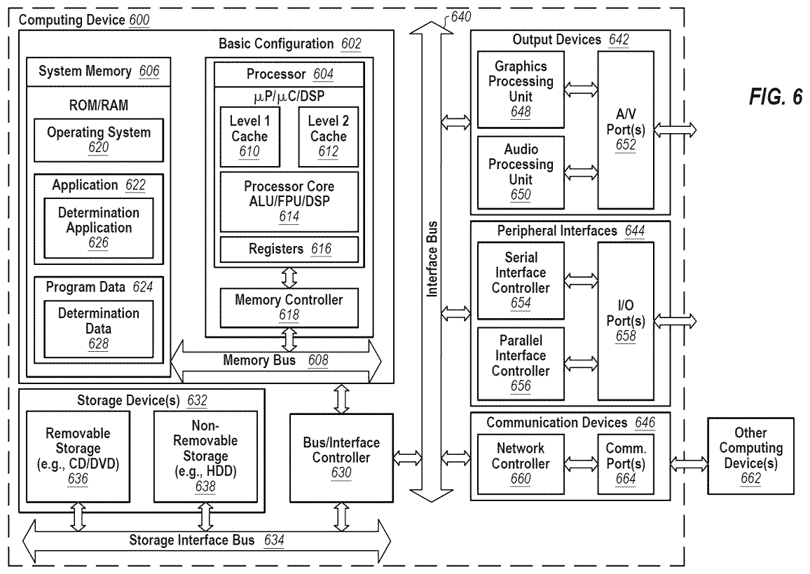

FIG. 6 shows an example computing device 600 (e.g., a computer) that may be arranged in some embodiments to perform the methods (or portions thereof) described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including, but not limited to: a microprocessor (μP), a microcontroller (μC), a digital signal processor (DSP), or any combination thereof. Processor 604 may include one or more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations, memory controller 618 may be an internal part of processor 604.

Depending on the desired configuration, system memory 606 may be of any type including, but not limited to: volatile memory (such as RAM), non-volatile memory (such as ROM, flash memory, etc.), or any combination thereof. System memory 606 may include an operating system 620, one or more applications 622, and program data 624. Application 622 may include a determination application 626 that is arranged to perform the operations as described herein, including those described with respect to methods described herein. The determination application 626 can obtain data, such as pressure, flow rate, and/or temperature, and then determine a change to the system to change the pressure, flow rate, and/or temperature.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method comprising computer-implemented steps as described herein.

Additionally, machine learning and deep learning techniques are utilized to assess the in vitro data and/or the in silico data. The invention provides methods that can be utilized to assess the translocation of agents across a cornea tissue (e.g., computer methods performed on data from in vitro or in silico source), and then determine whether the agent translocated sufficiently for a treatment or a toxicity to the fetus. The invention includes methods, system, apparatus, computer program product, among others, to carry out the following protocols, such as for generating a predicted biological data for a subject cornea based on the real in vitro biological data or in silico biological data. The predicted biological data from a model can be based on a perturbation or setting of at least one attribute of the subject for the in vitro or in silico data. The predicted biological data can be based on a simulation by a computer program for the simulated cornea model.

The methods can include receiving an in vitro data derived from in vitro tissue model. Based on the in vitro data, the method can include providing input vectors to a machine learning platform. The machine learning platform processes the input vectors in order to generate output that includes a generated in silico data of a simulated cornea model.

In some embodiments, the machine learning platform comprises one or more deep neural networks. In some aspects, the machine learning platform comprises one or generative adversarial networks. In some aspects, the machine learning platform comprises an adversarial autoencoder architecture.

Deep neural networks (DNNs) are computer system architectures that have recently been created for complex data processing and artificial intelligence (AI). DNNs are machine learning models that employ more than one hidden layer of nonlinear computational units to predict outputs for a set of received inputs. DNNs can be provided in various configurations for various purposes, and continue to be developed to improve performance and predictive ability.

In some embodiments, the executed method performed by the computer program product further comprises further training a model with reinforced leaning. The reinforced learning produces the reconstructed objects having a defined characteristic. In some aspects, the training of the trained model with the reinforced learning includes: discarding the object encoder; fixing weights of all layers of the object decoder except for a first layer of the object decoder; performing the following steps until convergence: estimate a mean and variance for each dimension of a previously obtained distribution of latent variables, the previously obtained distribution of latent variables being defined as a learnable prior; obtain an exploration latent variable for each dimension from outside of the latent variables produced by the encoder; pass the exploration latent variable through a decoder to obtain a reconstructed object based on the exploration latent variable; compute rewards for the reconstructed object based on at least one defined reward; and apply a single gradient ascent step to maximize a total reward with respect to a parameter of the learned prior and first layer of the decoder.

An autoencoder (AE) is a type of deep neural network (DNN) used in unsupervised learning for efficient information coding. The purpose of an AE is to learn a representation (e.g., encoding) of objects. An AE contains an encoder part, which is a DNN that transforms the input information from the input layer to the latent representation (e.g., latent code), and includes a decoder part, which uses the latent representation and decodes an original object with the output layer having the same dimensionality as the input for the encoder. Often, a use of an AE is for learning a representation or encoding for a set of data. An AE learns to compress data from the input layer into a short code, and then un-compress that code into something that closely matches the original data.

Generative Adversarial Networks (GANs) are structured probabilistic models that can be used to generate data. GANs can be used to generate data (e.g., a molecule) similar to the dataset (e.g., molecular library) GANs are trained on. A GAN can include two separate modules, which are DNN architectures called: (1) discriminator and (2) generator. The discriminator estimates the probability that a generated product comes from the real dataset, by working to compare a generated product to an original example, and is optimized to distinguish a generated product from the original example. The generator outputs generated products based on the original examples. The generator is trained to generate products that are as real as possible compared to an original example. The generator tries to improve its output in the form of a generated product until the discriminator is unable to distinguish the generated product from the real original example.

Adversarial Autoencoders (AAEs) are probabilistic AEs that use GANs to perform variational inference. AAEs are DNN-based architectures in which latent representations are forced to follow some prior distribution via the discriminator.

A conditional architecture may be considered a supervised architecture because the processing is supervised by the condition. As such, the conditional architecture may be configured for generating objects that match a specific condition (e.g., property of translocation of a molecule through the cornea). In some applications, a conditional model can take values of conditions into account, even if the values of conditions are only partially known. During the generation process, the conditional architecture may only have a few conditions that are specified, and thereby the rest of the conditions can take arbitrary values, at least initially.

Modern deep generative models are usually separated into two main categories. The first category is related to Generative Adversarial Networks (GAN), and the second one corresponds to models similar to Variational Autoencoders (VAE). Generative Adversarial Network is a model consisting of two networks—a generator and a discriminator. The generator produces a set of novel objects, while the discriminator tries to distinguish them from real ones. Variational and Adversarial Autoencoders build an autoencoder model with a regularizer that shapes the latent space. GANs produce state-of-the-art samples in specific domains, but Autoencoder-based approach allows training with discrete data and prevents the mode collapse problem.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety. This reference specifically incorporates U.S. Pat. Nos. 18/511,347 and 18/511,367, and the following:
1. Reinstein, D. Z., Archer, T. J., Gobbe, M., Silverman, R. H. & Coleman, D. J. Epithelial Thickness in the Normal Cornea: Three-dimensional Display With Very High Frequency Ultrasound. J Refract Surg 24, 571-581 (2008).
2. Reinstein, D. Z., Archer, T. J., Gobbe, M., Silverman, R. H. & Jackson Coleman, D. Stromal Thickness in the Normal Cornea: Three-dimensional Display With Artemis Very High-frequency Digital Ultrasound. J Refract Surg 25, 776-786 (2009).
3. Zavala, J., López Jaime, G. R., Rodríguez Barrientos, C. A. & Valdez-Garcia, J. Corneal endothelium: developmental strategies for regeneration. Eye 27, 579-588 (2013).
4. Tsonis, P. Animal Models in Eye Research. (Academic Press, 2008).

1. A microfluidic in vitro cornea device, comprising:
a tear flow chamber;
a stromal chamber adjacent to and porously coupled with the tear flow chamber;
an endothelial chamber adjacent to and porously coupled with the stromal chamber;
an aqueous humor chamber adjacent to and porously coupled with the endothelial chamber;
wherein a first porous wall is positioned between the tear flow chamber and the stromal chamber, a second porous wall is positioned between the stromal chamber and the endothelial chamber, and a third porous wall is positioned between the endothelial chamber and the aqueous humor chamber,
which is configured as a microfluidic in vitro model of a cornea.

2. The microfluidic in vitro cornea device of claim 1, wherein:
the tear flow chamber is shaped as a microfluidic arced structure that is configured to be coupled to a tear flow fluidic network with one or more pumps and optionally one or more media reservoirs; and
the aqueous humor chamber is shaped as a microfluidic domed structure that is configured to be coupled to an aqueous humor circulation fluidic network with one or more pumps and optionally one or more media reservoirs.

3. The microfluidic in vitro cornea device of claim 2, wherein at least one of:
the stromal chamber is shaped as a microfluidic arced structure, which may optionally be configured to be coupled to a stroma fluidic network with one or more pumps and optionally one or more media reservoirs; or
the endothelial chamber is shaped as a microfluidic arced structure, which may optionally be configured to be coupled to an endothelium fluidic network with one or more pumps and optionally one or more media reservoirs.

4. The microfluidic in vitro cornea device of claim 1, comprising in order:
the tear flow chamber;
the first porous wall;
the stromal chamber;
the second porous wall;
the endothelial chamber;
the third porous wall; and
the aqueous humor chamber.

5. The microfluidic in vitro cornea device of claim 1, comprising in order:
the tear film chamber having a width in a range from 20 microns to 500 microns;
a first porous wall having a width in a range from 20 microns to 100 microns;
the stromal chamber having a width in a range from 100 microns to 1000 microns;
a second porous wall having a width in a range from 20 microns to 100 microns;
the endothelial chamber having a width in a range from 10 microns to 200 microns;
a third porous wall having a width in a range from 20 microns to 100 microns;
the aqueous humor chamber having an apex width om a range from 100 microns to 5000 microns; and
a height the tear film chamber, stromal chamber, endothelial chamber, and aqueous humor chamber in a range from 10 microns to 1000 microns.

6. The microfluidic in vitro cornea device of claim 5, comprising in order:
the tear film chamber having an arc length in a range from 5 millimeters to 50 millimeters;
a first porous wall having an arc length in a range from 5 millimeters to 50 millimeters;
the stromal chamber having an arc length in a range from 5 millimeters to 50 millimeters;
a second porous wall having an arc length in a range from 5 millimeters to 50 millimeters;
the endothelium chamber having an arc length in a range from 5 millimeters to 50 millimeters;
a third porous wall having an arc length in a range from 5 millimeters to 50 millimeters; and
the aqueous humor chamber having a length in a range from 5 millimeters to 50 millimeters.

7. The microfluidic in vitro cornea device of claim 6, wherein each porous wall includes a plurality of pore channels that have a width that ranges from about 3 microns to about 8 microns and a height that ranges from about 6 microns to about 10 microns.

8. The microfluidic in vitro cornea device of claim 7, wherein each pore channel is spaced from about 25 microns to about 75 microns apart from another pore channel.

9. The microfluidic in vitro cornea device of claim 8, wherein the aqueous humor chamber includes a plurality of support posts that are configured to provide structural stability under physiological fluidic flow and pressure.

10. The microfluidic in vitro cornea device of claim 9, further comprising a lid, wherein the lid is at least partially supported by the support posts.

11. The microfluidic in vitro cornea device of claim 1, wherein:
the tear flow chamber includes epithelial cells;
the stromal chamber includes fibroblast cells;
the endothelial chamber includes endothelial cells; and
the aqueous humor chamber includes endothelial cells or is devoid of cells.

12. The microfluidic in vitro cornea device of claim 11,
the epithelial cells are primary human corneal epithelial cells;
the fibroblast cells are primary human corneal fibroblasts; and
the endothelial cells are immortalized human corneal endothelial cells.

13. The microfluidic in vitro cornea device of claim 12, comprising a co-culture or tri-culture of the epithelial cells, fibroblast cells, and endothelial cells.

14. The microfluidic in vitro cornea device of claim 1, comprising at least one viewing window into at least one of the tear flow chamber, stromal chamber, ocular endothelial chamber, or aqueous humor chamber.

15. A microfluidic in vitro cornea system comprising:
the microfluidic in vitro cornea device of claim 1; and
at least one pump configured for pumping fluid through the microfluidic in vitro cornea device in a closed loop or single pass and in unidirectional or bidirectional flow.

16. A microfluidic in vitro cornea system comprising:
the microfluidic in vitro cornea device of claim 1;
at least one camera device configured to be positioned to image at least one of the tear flow chamber, stromal chamber, endothelial chamber, or aqueous humor chamber; and
a computing system operably coupled with the at least one camera device to receive image data.

17. The microfluidic in vitro cornea system of claim 16, wherein the computing system is configured to obtain data from the at least one camera device and determine at least one trans-cornea transport barrier property of the microfluidic in vitro cornea device or at least one trans-cornea transport property of a test agent, wherein the trans-cornea transport barrier property is a measurement of inhibition of transport of a test agent across a corneal barrier and the trans-cornea transport property of the test agent is a measurement of traversal of the test agent across the corneal barrier.

18. The microfluidic in vitro cornea system of claim 17, wherein the computing system includes one or more computer-readable media storing instructions that when executed cause operations that determine the at least one trans-cornea transport barrier property of the microfluidic in vitro cornea device or the at least one trans-cornea transport property of a test agent.

19. A method of studying a cornea, comprising:
providing the microfluidic in vitro cornea device of claim 1 having epithelial cells in the tear flow chamber, stroma cells in the stromal chamber, and endothelial cells in the endothelial chamber;
determining a first condition of the microfluidic in vitro cornea device at a first time point;
determining a second condition of the in vitro cornea device at a subsequent time point; and
determining a change in condition of the in vitro cornea device from the first condition to the second condition.

20. The method of studying the cornea of claim 19, further comprising at least one of:
measuring a barrier function property of a corneal barrier, the corneal barrier including epithelial cells in the tear flow chamber, fibroblasts in the stromal chamber, and endothelial cells in the endothelial chamber;
imaging the tear flow chamber, stromal chamber, endothelial chamber, or aqueous humor chamber through a viewing window of the device;
viewing images in real time of the tear flow chamber, stromal chamber, endothelial chamber, or aqueous humor chamber through a display screen of a computing system;
measuring transport across the corneal barrier of at least one of nutrients, xenobiotics, small molecules, lipids, liposomes, polymers, particles, toxins, antibodies, or combinations thereof.

21. A method of studying transport of a test agent across a corneal barrier, comprising:
providing the microfluidic in vitro cornea device of claim 1 having epithelial cells in the tear flow chamber, fibroblast cells in the stromal chamber, and endothelial cells in the endothelial chamber;
providing a test agent to an input chamber selected from the tear flow chamber or aqueous humor chamber; and
monitoring trans-corneal transport of the test agent.

22. The method of studying transport of the test agents of claim 21, further comprising at least one of:
determining an amount of test agent crossing the corneal barrier and comparing the amount of test agent that crossed the corneal barrier with an administered amount of the test agent introduced into the microfluidic in vitro cornea device;
sampling the aqueous humor chamber for the test agent and quantifying the transport of the test agent across the corneal barrier into the aqueous humor chamber; or
sampling the tear flow chamber for the test agent and quantifying the transport of the test agent across the corneal barrier into the tear flow chamber.

23. The method of studying transport of the test agents of claim 21, further comprising evaluating barrier function of the corneal barrier:
injecting a plurality of different test agents having a plurality of different sizes into the tear flow chamber;
imaging the in vitro cornea device;
analyzing images of the in vitro cornea device to identify the plurality of different test agents; and
determining a size of test agent or size range of test agent of the plurality of test agents located in the tear flow chamber, stromal chamber, endothelial chamber, and/or aqueous humor chamber.

24. The method of studying transport of the test agents of claim 21, further comprising determining at least one of:
a size of test agent or size range of test agents capable of transporting from the tear flow chamber across the corneal barrier into the aqueous humor chamber;
a lipophilicity of test agent or lipophilicity range of test agents capable of transporting from the tear flow chamber across the stromal chamber and endothelial chamber into the aqueous humor chamber; or
a physiological charge of test agent or charge range capable of transporting the tear flow chamber across the stromal chamber and endothelial chamber into the aqueous humor chamber.

25. The method of studying transport of the test agents of claim 21, further comprising evaluating permeability of the in vitro cornea device by:
injecting one or more test agents into the tear flow chamber;
imaging the microfluidic in vitro cornea device;
analyzing images of the microfluidic in vitro cornea device to identify locations of the test agent at defined time points, and optionally determine amounts of each test agent in each chamber; and
determining a permeability of the in vitro cornea device for the one or more test agents.

26. The method of studying transport of the test agents of claim 21, further comprising determining a permeability index as a ratio of optical intensity measurements of the tear flow chamber with the aqueous humor chamber.

27. The method of studying transport of the test agents of claim 21, further comprising evaluating whether the test agent modifies permeability or structural integrity or morphology of the corneal barrier by:
determining an initial value of a first property of the corneal barrier;
introducing the test agent into the microfluidic in vitro cornea device;
determining a subsequent value of the first property of the corneal barrier; and
determining a difference between the initial value and the subsequent value of the first property of the corneal barrier, wherein the corneal barrier includes at least the stromal chamber and the epithelial cells of the tear flow chamber, and optionally endothelial cells in the endothelial chamber.

28. The method of studying transport of the test agents of claim 27, further comprising determining a health consequence of the test agent modulating the corneal barrier by correlating the difference between the initial value and the subsequent value and a phenotypic state, which phenotypic state may or may not be a disease state or disorder state.
